SEQUENCE LISTING (291 sequences)

SEQ ID NO: 1 - protein, 245 aa, Homo sapiens
  MISC_FEATURE: Chain A of C1q
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
  MISC_FEATURE: Chain B of C1q
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
  MISC_FEATURE: chain C of C1q
  MISC_FEATURE: Chain C of C1q
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 4 - protein, 7 aa, Homo sapiens
  MISC_FEATURE: synthetic
GSPGNIK

SEQ ID NO: 5 - protein, 11 aa, Homo sapiens
  MISC_FEATURE: synthetic
DQPRPAFSAIR

SEQ ID NO: 6 - protein, 28 aa, Homo sapiens
  MISC_FEATURE: synthetic
NPPMGGNVVIFDTVITNQEEPYQNHSGR

SEQ ID NO: 7 - protein, 30 aa, Homo sapiens
  MISC_FEATURE: synthetic
FVCTVPGYYYFTFQVLSQWEICLSIVSSSR

SEQ ID NO: 8 - protein, 30 aa, Homo sapiens; identical to SEQ ID NO: 7
  MISC_FEATURE: synthetic

SEQ ID NO: 9 - protein, 10 aa, Homo sapiens
  MISC_FEATURE: synthetic
SLGFCDTTNK

SEQ ID NO: 10 - protein, 10 aa, Homo sapiens
  MISC_FEATURE: synthetic
SLGFCDTTNK

SEQ ID NO: 11 - protein, 24 aa, Homo sapiens
  MISC_FEATURE: synthetic
GLFQVVSGGMVLQLQQGDQVWVEK

SEQ ID NO: 12 - protein, 22 aa, Homo sapiens
  MISC_FEATURE: synthetic
GHIYQGSEADSVFSGFLIFPSA

SEQ ID NO: 13 - protein, 5 aa, Homo sapiens
  MISC_FEATURE: synthetic
EDLCR

SEQ ID NO: 14 - protein, 5 aa, Homo sapiens
  MISC_FEATURE: synthetic
APDGK

SEQ ID NO: 15 - protein, 8 aa, Homo sapiens
  MISC_FEATURE: synthetic
GEAGRPGR

SEQ ID NO: 16 - protein, 12 aa, Homo sapiens
  MISC_FEATURE: synthetic
GEQGEPGAPGIR

SEQ ID NO: 17 - protein, 13 aa, Homo sapiens
  MISC_FEATURE: synthetic
VGYPGPSGPLGAR

SEQ ID NO: 18 - protein, 4 aa, Homo sapiens
  MISC_FEATURE: synthetic
ATQK

SEQ ID NO: 19 - protein, 5 aa, Homo sapiens
  MISC_FEATURE: synthetic
DQTIR

SEQ ID NO: 20 - protein, 4 aa, Homo sapiens
  MISC_FEATURE: synthetic
FTCK

SEQ ID NO: 21 - protein, 14 aa, Homo sapiens
  MISC_FEATURE: synthetic
VPGLYYFTYHASSR

SEQ ID NO: 22 - protein, 9 aa, Homo sapiens
  MISC_FEATURE: synthetic
GNLCVNLMR

SEQ ID NO: 23 - protein, 9 aa, Homo sapiens
  MISC_FEATURE: synthetic
GNLCVNLMR

SEQ ID NO: 24 - protein, 22 aa, Homo sapiens
  MISC_FEATURE: synthetic
VVTFCDYAYNTFQVTTGGMVLK

SEQ ID NO: 25 - protein, 22 aa, Homo sapiens
  MISC_FEATURE: synthetic
VVTFCDYAYNTFQVTTGGMVLK

SEQ ID NO: 26 - protein, 14 aa, Homo sapiens
  MISC_FEATURE: synthetic
LEQGENVFLQATDK

SEQ ID NO: 27 - protein, 24 aa, Homo sapiens
  MISC_FEATURE: synthetic
NSLLGMEGANSIFSGFLLFPDMEA

SEQ ID NO: 28 - protein, 32 aa, Homo sapiens
  MISC_FEATURE: synthetic
QLSCTGPPAIPGIPGIPGTPGPDGQPGTPGIK

SEQ ID NO: 29 - protein, 32 aa, Homo sapiens; identical to SEQ ID NO: 28
  MISC_FEATURE: synthetic

SEQ ID NO: 30 - protein, 15 aa, Homo sapiens
  MISC_FEATURE: synthetic
GLPGLAGDHGEFGEK

SEQ ID NO: 31 - protein, 11 aa, Homo sapiens
  MISC_FEATURE: synthetic
GDPGIPGNPGK

SEQ ID NO: 32 - protein, 6 aa, Homo sapiens
  MISC_FEATURE: synthetic
GPMGPK

SEQ ID NO: 33 - protein, 12 aa, Homo sapiens
  MISC_FEATURE: synthetic
GGPGAPGAPGPK

SEQ ID NO: 34 - protein, 9 aa, Homo sapiens
  MISC_FEATURE: synthetic
FQSVFTVTR

SEQ ID NO: 35 - protein, 13 aa, Homo sapiens
  MISC_FEATURE: synthetic
QTHQPPAPNSLIR

SEQ ID NO: 36 - protein, 18 aa, Homo sapiens
  MISC_FEATURE: synthetic
FNAVLTNPQGDYDTSTGK

SEQ ID NO: 37 - protein, 23 aa, Homo sapiens
  MISC_FEATURE: synthetic
VPGLYYFVYHASHTANLCVLLYR

SEQ ID NO: 38 - protein, 23 aa, Homo sapiens
  MISC_FEATURE: synthetic
VPGLYYFVYHASHTANLCVLLYR

SEQ ID NO: 39 - protein, 10 aa, Homo sapiens
  MISC_FEATURE: synthetic
VVTFCGHTSK

SEQ ID NO: 40 - protein, 10 aa, Homo sapiens
  MISC_FEATURE: synthetic
VVTFCGHTSK

SEQ ID NO: 41 - protein, 12 aa, Homo sapiens
  MISC_FEATURE: synthetic
TNQVNSGGVLLR

SEQ ID NO: 42 - protein, 35 aa, Homo sapiens
  MISC_FEATURE: syynthetic
LQVGEEVWLAVNDYYDMVGIQGSDSVFSGFLLFPD

SEQ ID NO: 43 - protein, 19 aa, Homo sapiens
  MISC_FEATURE: synthetic
NTGCYGIPGMPGLPGAPGK

SEQ ID NO: 44 - protein, 19 aa, Homo sapiens
  MISC_FEATURE: synthetic
NTGCYGIPGMPGLPGAPGK

SEQ ID NO: 45 - protein, 10 aa, Homo sapiens
  MISC_FEATURE: synthetic
DGYDGLPGPK

SEQ ID NO: 46 - protein, 12 aa, Homo sapiens
  MISC_FEATURE: synthetic
GEPGIPAIPGIR

SEQ ID NO: 47 - protein, 11 aa, Homo sapiens
  MISC_FEATURE: synthetic
GEPGLPGHPGK

SEQ ID NO: 48 - protein, 27 aa, Homo sapiens
  MISC_FEATURE: synthetic
NGPMGPPGMPGVPGPMGIPGEPGEEGR

SEQ ID NO: 49 - protein, 500 aa, Homo sapiens
  MISC_FEATURE: synthetic
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 50 - protein, 10 aa, Homo sapiens
  MISC_FEATURE: synthetic
DFTCVHQALK

SEQ ID NO: 51 - protein, 10 aa, Homo sapiens
  MISC_FEATURE: synthetic
DFTCVHQALK

SEQ ID NO: 52 - protein, 5 aa, Homo sapiens
  MISC_FEATURE: synthetic
GFTTK

SEQ ID NO: 53 - protein, 17 aa, Homo sapiens
  MISC_FEATURE: synthetic
GVTSVSQIFHSPDLAIR

SEQ ID NO: 54 - protein, 18 aa, Homo sapiens
  MISC_FEATURE: synthetic
NPNATSSSSQDPESLQDR

SEQ ID NO: 55 - protein, 8 aa, Homo sapiens
  MISC_FEATURE: synthetic
DTFVNASR

SEQ ID NO: 56 - protein, 8 aa, Homo sapiens
  MISC_FEATURE: synthetic
TLYSSSPR

SEQ ID NO: 57 - protein, 19 aa, Homo sapiens
  MISC_FEATURE: synthetic
VLSNNSDANLELINTWVAK

SEQ ID NO: 58 - protein, 5 aa, Homo sapiens
  MISC_FEATURE: synthetic
NTNNK

SEQ ID NO: 59 - protein, 10 aa, Homo sapiens
  MISC_FEATURE: synthetic
LLDSLPSDTR

SEQ ID NO: 60 - protein, 6 aa, Homo sapiens
  MISC_FEATURE: synthetic
TTFDPK

SEQ ID NO: 61 - protein, 7 aa, Homo sapiens
  MISC_FEATURE: synthetic
MEPFHFK

SEQ ID NO: 62 - protein, 7 aa, Homo sapiens
  MISC_FEATURE: synthetic
VPMMNSK

SEQ ID NO: 63 - protein, 12 aa, Homo sapiens
  MISC_FEATURE: synthetic
YPVAHFIDQTLK

SEQ ID NO: 64 - protein, 21 aa, Homo sapiens
  MISC_FEATURE: synthetic
VGQLQLSHNLSLVILVPQNLK

SEQ ID NO: 65 - protein, 14 aa, Homo sapiens
  MISC_FEATURE: synthetic
LEDMEQALSPSVFK

SEQ ID NO: 66 - protein, 5 aa, Homo sapiens
  MISC_FEATURE: synthetic
AIMEK

SEQ ID NO: 67 - protein, 5 aa, Homo sapiens
  MISC_FEATURE: synthetic
LEMSK

SEQ ID NO: 68 - protein, 10 aa, Homo sapiens
  MISC_FEATURE: synthetic
FQPTLLTLPR

SEQ ID NO: 69 - protein, 13 aa, Homo sapiens
  MISC_FEATURE: synthetic
VTTSQDMLSIMEK

SEQ ID NO: 70 - protein, 4 aa, Homo sapiens
  MISC_FEATURE: synthetic
GEGK

SEQ ID NO: 71 - protein, 51 aa, Homo sapiens
  MISC_FEATURE: synthetic
LEFFDFSYDLNLCGLTEDPDLQVSAMQHQTVLELTETGVEAAAASAISVAR

SEQ ID NO: 72 - protein, 21 aa, Homo sapiens
  MISC_FEATURE: synthetic
TLLVFEVQQPFLFVLWDQQHK

SEQ ID NO: 73 - protein, 7 aa, Homo sapiens
  MISC_FEATURE: synthetic
FPVFMGR

SEQ ID NO: 74 - protein, 5 aa, Homo sapiens
  MISC_FEATURE: synthetic
VYDPR

SEQ ID NO: 75 - protein, 25 aa, Homo sapiens
  MISC_FEATURE: synthetic
MLFVEPILEVSSLPTTNSTTNSATK

SEQ ID NO: 76 - protein, 1698 aa, Homo sapiens
  MISC_FEATURE: synthetic
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 77 - protein, 18 aa, Homo sapiens
  MISC_FEATURE: synthetic
GCGEQTMIYLAPTLAASR

SEQ ID NO: 78 - protein, 18 aa, Homo sapiens
  MISC_FEATURE: synthetic
GCGEQTMIYLAPTLAASR

SEQ ID NO: 79 - protein, 4 aa, Homo sapiens
  MISC_FEATURE: synthetic
YLDK

SEQ ID NO: 80 - protein, 12 aa, Homo sapiens
  MISC_FEATURE: synthetic
TEQWSTLPPETK

SEQ ID NO: 81 - protein, 9 aa, Homo sapiens
  MISC_FEATURE: synthetic
DHAVDLIQK

SEQ ID NO: 82 - protein, 4 aa, Homo sapiens
  MISC_FEATURE: synthetic
GYMR

SEQ ID NO: 83 - protein, 11 aa, Homo sapiens
  MISC_FEATURE: synthetic
ADGSYAAWLSR

SEQ ID NO: 84 - protein, 12 aa, Homo sapiens
  MISC_FEATURE: synthetic
GSSTWLTAFVLK

SEQ ID NO: 85 - protein, 15 aa, Homo sapiens
  MISC_FEATURE: synthetic
VLSLAQEQVGGSPEK

SEQ ID NO: 86 - protein, 27 aa, Homo sapiens
  MISC_FEATURE: synthetic
LQETSNWLLSQQQADGSFQDLSPVIHR

SEQ ID NO: 87 - protein, 7 aa, Homo sapiens
  MISC_FEATURE: synthetic
VEASISK

SEQ ID NO: 88 - protein, 8 aa, Homo sapiens
  MISC_FEATURE: synthetic
ASSFLGEK

SEQ ID NO: 89 - protein, 22 aa, Homo sapiens
  MISC_FEATURE: synthetic
ASAGLLGAHAAAITAYALTLTK

SEQ ID NO: 90 - protein, 38 aa, Homo sapiens
  MISC_FEATURE: synthetic
GVAHNNLMAMAQETGDNLYWGSVTGSQSNAVSPTPAPR

SEQ ID NO: 91 - protein, 30 aa, Homo sapiens
  MISC_FEATURE: synthetic
NPSDPMPQAPALWIETTAYALLHLLLHEGK

SEQ ID NO: 92 - protein, 13 aa, Homo sapiens
  MISC_FEATURE: synthetic
AEMADQAAAWLTR

SEQ ID NO: 93 - protein, 9 aa, Homo sapiens
  MISC_FEATURE: synthetic
QGSFQGGFR

SEQ ID NO: 94 - protein, 25 aa, Homo sapiens
  MISC_FEATURE: synthetic
STQDTVIALDALSAYWIASHTTEER

SEQ ID NO: 95 - protein, 11 aa, Homo sapiens
  MISC_FEATURE: synthetic
GLNVTLSSTGR

SEQ ID NO: 96 - protein, 4 aa, Homo sapiens
  MISC_FEATURE: synthetic
NGFK

SEQ ID NO: 97 - protein, 9 aa, Homo sapiens
  MISC_FEATURE: synthetic
SHALQLNNR

SEQ ID NO: 98 - protein, 13 aa, Homo sapiens
  MISC_FEATURE: synthetic
GLEEELQFSLGSK

SEQ ID NO: 99 - protein, 6 aa, Homo sapiens
  MISC_FEATURE: synthetic
VGGNSK

SEQ ID NO: 100 - protein, 8 aa, Homo sapiens
  MISC_FEATURE: synthetic
TYNVLDMK

SEQ ID NO: 101 - protein, 14 aa, Homo sapiens
  MISC_FEATURE: synthetic
NTTCQDLQIEVTVK

SEQ ID NO: 102 - protein, 14 aa, Homo sapiens
  MISC_FEATURE: synthetic
NTTCQDLQIEVTVK

SEQ ID NO: 103 - protein, 24 aa, Homo sapiens
  MISC_FEATURE: synthetic
GHVEYTMEANEDYEDYEYDELPAK

SEQ ID NO: 104 - protein, 18 aa, Homo sapiens
  MISC_FEATURE: synthetic
DDPDAPLQPVTPLQLFEG

SEQ ID NO: 105 - protein, 6 aa, Homo sapiens
  MISC_FEATURE: synthetic
NVNFQK

SEQ ID NO: 106 - protein, 5 aa, Homo sapiens
  MISC_FEATURE: synthetic
AINEK

SEQ ID NO: 107 - protein, 10 aa, Homo sapiens
  MISC_FEATURE: synthetic
LGQYASPTAK

SEQ ID NO: 108 - protein, 8 aa, Homo sapiens
  MISC_FEATURE: synthetic
CCQDGVTR

SEQ ID NO: 109 - protein, 5 aa, Homo sapiens
  MISC_FEATURE: synthetic
LPMMR

SEQ ID NO: 110 - protein, 5 aa, Homo sapiens
  MISC_FEATURE: synthetic
SCEQR

SEQ ID NO: 111 - protein, 7 aa, Homo sapiens
  MISC_FEATURE: synthetic
VQQPDCR

SEQ ID NO: 112 - protein, 7 aa, Homo sapiens
  MISC_FEATURE: synthetic
VQQPDCR

SEQ ID NO: 113 - protein, 14 aa, Homo sapiens
  MISC_FEATURE: synthetic
EPFLSCCQFAESLR

SEQ ID NO: 114 - protein, 7 aa, Homo sapiens
  MISC_FEATURE: synthetic
GQAGLQR

SEQ ID NO: 115 - protein, 19 aa, Homo sapiens
  MISC_FEATURE: synthetic
ALEILQEEDLIDEDDIPVR

SEQ ID NO: 116 - protein, 10 aa, Homo sapiens
  MISC_FEATURE: synthetic
SFFPENWLWR

SEQ ID NO: 117 - protein, 6 aa, Homo sapiens
  MISC_FEATURE: syynthetic
VETVDR

SEQ ID NO: 118 - protein, 24 aa, Homo sapiens
  MISC_FEATURE: synthetic
FQILTLWLPDSLTTWEIHGLSLSK

SEQ ID NO: 119 - protein, 11 aa, Homo sapiens
  MISC_FEATURE: synthetic
GLCVATPVQLR

SEQ ID NO: 120 - protein, 11 aa, Homo sapiens
  MISC_FEATURE: synthetic
GLCVATPVQLR

SEQ ID NO: 121 - protein, 7 aa, Homo sapiens
  MISC_FEATURE: synthetic
EFHLHLR

SEQ ID NO: 122 - protein, 16 aa, Homo sapiens
  MISC_FEATURE: synthetic
FEQLELRPVLYNYLDK

SEQ ID NO: 123 - protein, 51 aa, Homo sapiens
  MISC_FEATURE: synthetic
NLTVSVHVSPVEGLCLAGGGGLAQQVLVPAGSARPVAFSVVPTAATAVSLK

SEQ ID NO: 124 - protein, 51 aa, Homo sapiens; identical to SEQ ID NO: 123
  MISC_FEATURE: synthetic

SEQ ID NO: 125 - protein, 4 aa, Homo sapiens
  MISC_FEATURE: synthetic
VVAR

SEQ ID NO: 126 - protein, 13 aa, Homo sapiens
  MISC_FEATURE: synthetic
GSFEFPVGDAVSK

SEQ ID NO: 127 - protein, 6 aa, Homo sapiens
  MISC_FEATURE: synthetic
EGAIHR

SEQ ID NO: 128 - protein, 13 aa, Homo sapiens
  MISC_FEATURE: synthetic
EELVYELNPLDHR

SEQ ID NO: 129 - protein, 23 aa, Homo sapiens
  MISC_FEATURE: synthetic
TLEIPGNSDPNMIPDGDFNSYVR

SEQ ID NO: 130 - protein, 26 aa, Homo sapiens
  MISC_FEATURE: synthetic
VTASDPLDTLGSEGALSPGGVASLLR

SEQ ID NO: 131 - protein, 14 aa, Homo sapiens
  MISC_FEATURE: synthetic
GPEVQLVAHSPWLK

SEQ ID NO: 132 - protein, 5 aa, Homo sapiens
  MISC_FEATURE: synthetic
DSLSR

SEQ ID NO: 133 - protein, 14 aa, Homo sapiens
  MISC_FEATURE: synthetic
TTNIQGINLLFSSR

SEQ ID NO: 134 - protein, 17 aa, Homo sapiens
  MISC_FEATURE: synthetic
GHLFLQTDQPIYNPGQR

SEQ ID NO: 135 - protein, 7 aa, Homo sapiens
  MISC_FEATURE: synthetic
VFALDQK

SEQ ID NO: 136 - protein, 19 aa, Homo sapiens
  MISC_FEATURE: synthetic
MRPSTDTITVMVENSHGLR

SEQ ID NO: 137 - protein, 25 aa, Homo sapiens
  MISC_FEATURE: synthetic
EVYMPSSIFQDDFVIPDISEPGTWK

SEQ ID NO: 138 - protein, 16 aa, Homo sapiens
  MISC_FEATURE: synthetic
FSDGLESNSSTQFEVK

SEQ ID NO: 139 - protein, 26 aa, Homo sapiens
  MISC_FEATURE: synthetic
LLLFSPSVVHLGVPLSVGVQLQDVPR

SEQ ID NO: 140 - protein, 9 aa, Homo sapiens
  MISC_FEATURE: synthetic
YVLPNFEVK

SEQ ID NO: 141 - protein, 25 aa, Homo sapiens
  MISC_FEATURE: synthetic
ITPGKPYILTVPGHLDEMQLDIQAR

SEQ ID NO: 142 - protein, 14 aa, Homo sapiens
  MISC_FEATURE: synthetic
YIYGKPVQGVAYVR

SEQ ID NO: 143 - protein, 9 aa, Homo sapiens
  MISC_FEATURE: synthetic
FGLLDEDGK

SEQ ID NO: 144 - protein, 4 aa, Homo sapiens
  MISC_FEATURE: synthetic
TFFR

SEQ ID NO: 145 - protein, 7 aa, Homo sapiens
  MISC_FEATURE: synthetic
GLESQTK

SEQ ID NO: 146 - protein, 12 aa, Homo sapiens
LVNGQSHISLSK

SEQ ID NO: 147 - protein, 12 aa, Homo sapiens
  MISC_FEATURE: synthetic
LNMGITDLQGLR

SEQ ID NO: 148 - protein, 36 aa, Homo sapiens
  MISC_FEATURE: synthetic
LYVAAAIIESPGGEMEEAELTSWYFVSSPFSLDLSK

SEQ ID NO: 149 - protein, 13 aa, Homo sapiens
  MISC_FEATURE: synthetic
EMSGSPASGIPVK

SEQ ID NO: 150 - protein, 55 aa, Homo sapiens
  MISC_FEATURE: synthetic
VSATVSSPGSVPEVQDIQQNTDGSGQVSIPIIIPQTISELQLSVSAGSPHPAIAR

SEQ ID NO: 151 - protein, 25 aa, Homo sapiens
  MISC_FEATURE: synthetic
LTVAAPPSGGPGFLSIERPDSRPPR

SEQ ID NO: 152 - protein, 10 aa, Homo sapiens
  MISC_FEATURE: synthetic
VGDTLNLNLR

SEQ ID NO: 153 - protein, 5 aa, Homo sapiens
  MISC_FEATURE: synthetic
GQVVK

SEQ ID NO: 154 - protein, 18 aa, Homo sapiens
  MISC_FEATURE: synthetic
AVGSGATFSHYYYMILSR

SEQ ID NO: 155 - protein, 8 aa, Homo sapiens
  MISC_FEATURE: synthetic
GQIVFMNR

SEQ ID NO: 156 - protein, 35 aa, Homo sapiens
  MISC_FEATURE: synthetic
TLTSVSVFVDHHLAPSFYFVAFYYHGDHPVANSLR

SEQ ID NO: 157 - protein, 11 aa, Homo sapiens
  MISC_FEATURE: synthetic
VDVQAGACEGK

SEQ ID NO: 158 - protein, 11 aa, Homo sapiens
  MISC_FEATURE: synthetic
VDVQAGACEGK

SEQ ID NO: 159 - protein, 9 aa, Homo sapiens
  MISC_FEATURE: synthetic
LELSVDGAK

SEQ ID NO: 160 - protein, 6 aa, Homo sapiens
  MISC_FEATURE: synthetic
NGESVK

SEQ ID NO: 161 - protein, 26 aa, Homo sapiens
  MISC_FEATURE: synthetic
LHLETDSLALVALGALDTALYAAGSK

SEQ ID NO: 162 - protein, 4 aa, Homo sapiens
  MISC_FEATURE: synthetic
NPSR

SEQ ID NO: 163 - protein, 9 aa, Homo sapiens
  MISC_FEATURE: synthetic
SHKPLNMGK

SEQ ID NO: 164 - protein, 41 aa, Homo sapiens
  MISC_FEATURE: synthetic
VFEAMNSYDLGCGPGGGDSALQVFQAAGLAFSDGDQWTLSR

SEQ ID NO: 165 - protein, 41 aa, Homo sapiens; identical to SEQ ID NO: 164
  MISC_FEATURE: synthetic

SEQ ID NO: 166 - protein, 8 aa, Homo sapiens
  MISC_FEATURE: synthetic
NNVPCSPK

SEQ ID NO: 167 - protein, 8 aa, Homo sapiens
  MISC_FEATURE: synthetic
NNVPCSPK

SEQ ID NO: 168 - protein, 5 aa, Homo sapiens
  MISC_FEATURE: synthetic
LSCPK

SEQ ID NO: 169 - protein, 5 aa, Homo sapiens
  MISC_FEATURE: synthetic
LSCPK

SEQ ID NO: 170 - protein, 9 aa, Homo sapiens
  MISC_FEATURE: synthetic
VDFTLSSER

SEQ ID NO: 171 - protein, 12 aa, Homo sapiens
  MISC_FEATURE: synthetic
DFALLSLQVPLK

SEQ ID NO: 172 - protein, 9 aa, Homo sapiens
  MISC_FEATURE: synthetic
SCGLHQLLR

SEQ ID NO: 173 - protein, 9 aa, Homo sapiens
  MISC_FEATURE: synthetic
SCGLHQLLR

SEQ ID NO: 174 - protein, 8 aa, Homo sapiens
  MISC_FEATURE: synthetic
VVEEQESR

SEQ ID NO: 175 - protein, 9 aa, Homo sapiens
  MISC_FEATURE: synthetic
VHYTVCIWR

SEQ ID NO: 176 - protein, 9 aa, Homo sapiens
  MISC_FEATURE: synthetic
VHYTVCIWR

SEQ ID NO: 177 - protein, 21 aa, Homo sapiens
  MISC_FEATURE: synthetic
VGLSGMAIADVTLLSGFHALR

SEQ ID NO: 178 - protein, 5 aa, Homo sapiens
  MISC_FEATURE: synthetic
ADLEK

SEQ ID NO: 179 - protein, 7 aa, Homo sapiens
  MISC_FEATURE: synthetic
LTSLSDR

SEQ ID NO: 180 - protein, 23 aa, Homo sapiens
  MISC_FEATURE: synthetic
YVSHFETEGPHVLLYFDSVPTSR

SEQ ID NO: 181 - protein, 31 aa, Homo sapiens
  MISC_FEATURE: synthetic
ECVGFEAVQEVPVGLVQPASATLYDYYNPER

SEQ ID NO: 182 - protein, 31 aa, Homo sapiens; identical to SEQ ID NO: 181
  MISC_FEATURE: synthetic

SEQ ID NO: 183 - protein, 10 aa, Homo sapiens
  MISC_FEATURE: synthetic
CSVFYGAPSK

SEQ ID NO: 184 - protein, 10 aa, Homo sapiens
  MISC_FEATURE: synthetic
CSVFYGAPSK

SEQ ID NO: 185 - protein, 17 aa, Homo sapiens
  MISC_FEATURE: synthetic
LLATLCSAEVCQCAEGK

SEQ ID NO: 186 - protein, 17 aa, Homo sapiens
  MISC_FEATURE: synthetic
LLATLCSAEVCQCAEGK

SEQ ID NO: 187 - protein, 4 aa, Homo sapiens
  MISC_FEATURE: synthetic
ALER

SEQ ID NO: 188 - protein, 7 aa, Homo sapiens
  MISC_FEATURE: synthetic
FACYYPR

SEQ ID NO: 189 - protein, 7 aa, Homo sapiens
  MISC_FEATURE: synthetic
FACYYPR

SEQ ID NO: 190 - protein, 8 aa, Homo sapiens
  MISC_FEATURE: synthetic
VEYGFQVK

SEQ ID NO: 191 - protein, 4 aa, Homo sapiens
  MISC_FEATURE: synthetic
AAFR

SEQ ID NO: 192 - protein, 5 aa, Homo sapiens
  MISC_FEATURE: synthetic
LFETK

SEQ ID NO: 193 - protein, 7 aa, Homo sapiens
  MISC_FEATURE: synthetic
AAANQMR

SEQ ID NO: 194 - protein, 4 aa, Homo sapiens
  MISC_FEATURE: synthetic
ASCR

SEQ ID NO: 195 - protein, 5 aa, Homo sapiens
  MISC_FEATURE: synthetic
LEPGK

SEQ ID NO: 196 - protein, 35 aa, Homo sapiens
  MISC_FEATURE: synthetic
EYLIMGLDGATYDLEGHPQYLLDSNSWIEEMPSER

SEQ ID NO: 197 - protein, 20 aa, Homo sapiens
  MISC_FEATURE: synthetic
AACAQLNDFLQEYGTQGCQV

SEQ ID NO: 198 - protein, 20 aa, Homo sapiens
  MISC_FEATURE: synthetic
AACAQLNDFLQEYGTQGCQV

SEQ ID NO: 199 - protein, 1663 aa, Homo sapiens
  MISC_FEATURE: C3
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 200 - protein, 15 aa, Homo sapiens
  MISC_FEATURE: synthetic
FVTVQATFGTQVVEK

SEQ ID NO: 201 - protein, 17 aa, Homo sapiens
  MISC_FEATURE: synthetic
VVLVSLQSGYLFIQTDK

SEQ ID NO: 202 - protein, 12 aa, Homo sapiens
  MISC_FEATURE: synthetic
TIYTPGSTVLYR

SEQ ID NO: 203 - protein, 7 aa, Homo sapiens
  MISC_FEATURE: synthetic
IFTVNHK

SEQ ID NO: 204 - protein, 6 aa, Homo sapiens
  MISC_FEATURE: synthetic
LLPVGR

SEQ ID NO: 205 - protein, 15 aa, Homo sapiens
  MISC_FEATURE: synthetic
TVMVNIENPEGIPVK

SEQ ID NO: 206 - protein, 29 aa, Homo sapiens
  MISC_FEATURE: synthetic
QDSLSSQNQLGVLPLSWDIPELVNMGQWK

SEQ ID NO: 207 - protein, 18 aa, Homo sapiens
  MISC_FEATURE: synthetic
AYYENSPQQVFSTEFEVK

SEQ ID NO: 208 - protein, 16 aa, Homo sapiens
  MISC_FEATURE: synthetic
EYVLPSFEVIVEPTEK

SEQ ID NO: 209 - protein, 13 aa, Homo sapiens
  MISC_FEATURE: synthetic
SPMYSIITPNILR

SEQ ID NO: 210 - protein, 8 aa, Homo sapiens
  MISC_FEATURE: synthetic
FYYIYNEK

SEQ ID NO: 211 - protein, 9 aa, Homo sapiens
  MISC_FEATURE: synthetic
GLEVTITAR

SEQ ID NO: 212 - protein, 5 aa, Homo sapiens
  MISC_FEATURE: synthetic
FLYGK

SEQ ID NO: 213 - protein, 17 aa, Homo sapiens
  MISC_FEATURE: synthetic
VEGTAFVIFGIQDGEQR

SEQ ID NO: 214 - protein, 14 aa, Homo sapiens
  MISC_FEATURE: synthetic
IPIEDGSGEVVLSR

SEQ ID NO: 215 - protein, 10 aa, Homo sapiens
  MISC_FEATURE: synthetic
VLLDGVQNPR

SEQ ID NO: 216 - protein, 7 aa, Homo sapiens
  MISC_FEATURE: synthetic
AEDLVGK

SEQ ID NO: 217 - protein, 21 aa, Homo sapiens
  MISC_FEATURE: synthetic
SLYVSATVILHSGSDMVQAER

SEQ ID NO: 218 - protein, 16 aa, Homo sapiens
  MISC_FEATURE: synthetic
SGIPIVTSPYQIHFTK

SEQ ID NO: 219 - protein, 24 aa, Homo sapiens
  MISC_FEATURE: synthetic
YFKPGMPFDLMVFVTNPDGSPAYR

SEQ ID NO: 220 - protein, 30 aa, Homo sapiens
  MISC_FEATURE: synthetic
LESEETMVLEAHDAQGDVPVTVTVHDFPGK

SEQ ID NO: 221 - protein, 22 aa, Homo sapiens
  MISC_FEATURE: synthetic
VPVAVQGEDTVQSLTQGDGVAK

SEQ ID NO: 222 - protein, 17 aa, Homo sapiens
  MISC_FEATURE: synthetic
LSINTHPSQKPLSITVR

SEQ ID NO: 223 - protein, 11 aa, Homo sapiens
  MISC_FEATURE: synthetic
QELSEAEQATR

SEQ ID NO: 224 - protein, 23 aa, Homo sapiens
  MISC_FEATURE: synthetic
TMQALPYSTVGNSNNYLHLSVLR

SEQ ID NO: 225 - protein, 16 aa, Homo sapiens
  MISC_FEATURE: synthetic
TELRPGETLNVNFLLR

SEQ ID NO: 226 - protein, 5 aa, Homo sapiens
  MISC_FEATURE: synthetic
AHEAK

SEQ ID NO: 227 - protein, 9 aa, Homo sapiens
  MISC_FEATURE: synthetic
YYTYLIMNK

SEQ ID NO: 228 - protein, 22 aa, Homo sapiens
  MISC_FEATURE: synthetic
EPGQDLVVLPLSITTDFIPSFR

SEQ ID NO: 229 - protein, 14 aa, Homo sapiens
  MISC_FEATURE: synthetic
LVAYYTLIGASGQR

SEQ ID NO: 230 - protein, 12 aa, Homo sapiens
  MISC_FEATURE: synthetic
EVVADSVWVDVK

SEQ ID NO: 231 - protein, 10 aa, Homo sapiens
  MISC_FEATURE: synthetic
DSCVGSLVVK

SEQ ID NO: 232 - protein, 10 aa, Homo sapiens
  MISC_FEATURE: synthetic
DSCVGSLVVK

SEQ ID NO: 233 - protein, 11 aa, Homo sapiens
  MISC_FEATURE: synthetic
QPVPGQQMTLK

SEQ ID NO: 234 - protein, 8 aa, Homo sapiens
  MISC_FEATURE: synthetic
IEGDHGAR

SEQ ID NO: 235 - protein, 7 aa, Homo sapiens
  MISC_FEATURE: synthetic
IWDVVEK

SEQ ID NO: 236 - protein, 11 aa, Homo sapiens
  MISC_FEATURE: synthetic
ADIGCTPGSGK

SEQ ID NO: 237 - protein, 24 aa, Homo sapiens
  MISC_FEATURE: synthetic
DYAGVFSDAGLTFTSSSGQQTAQR

SEQ ID NO: 238 - protein, 10 aa, Homo sapiens
  MISC_FEATURE: synthetic
AELQCPQPAA

SEQ ID NO: 239 - protein, 10 aa, Homo sapiens
  MISC_FEATURE: synthetic
AELQCPQPAA

SEQ ID NO: 240 - protein, 7 aa, Homo sapiens
  MISC_FEATURE: synthetic
LVLSSEK

SEQ ID NO: 241 - protein, 21 aa, Homo sapiens
  MISC_FEATURE: synthetic
TVLTPATNHMGNVTFTIPANR

SEQ ID NO: 242 - protein, 16 aa, Homo sapiens
  MISC_FEATURE: synthetic
SNLDEDIIAEENIVSR

SEQ ID NO: 243 - protein, 15 aa, Homo sapiens
  MISC_FEATURE: synthetic
SEFPESWLWNVEDLK

SEQ ID NO: 244 - protein, 4 aa, Homo sapiens
  MISC_FEATURE: synthetic
EPPK

SEQ ID NO: 245 - protein, 6 aa, Homo sapiens
  MISC_FEATURE: synthetic
NGISTK

SEQ ID NO: 246 - protein, 16 aa, Homo sapiens
  MISC_FEATURE: synthetic
DSITTWEILAVSMSDK

SEQ ID NO: 247 - protein, 21 aa, Homo sapiens
  MISC_FEATURE: synthetic
GICVADPFEVTVMQDFFIDLR

SEQ ID NO: 248 - protein, 21 aa, Homo sapiens
  MISC_FEATURE: synthetic
GICVADPFEVTVMQDFFIDLR

SEQ ID NO: 249 - protein, 7 aa, Homo sapiens
  MISC_FEATURE: synthetic
LPYSVVR

SEQ ID NO: 250 - protein, 7 aa, Homo sapiens
  MISC_FEATURE: synthetic
NEQVEIR

SEQ ID NO: 251 - protein, 7 aa, Homo sapiens
  MISC_FEATURE: synthetic
AVLYNYR

SEQ ID NO: 252 - protein, 6 aa, Homo sapiens
  MISC_FEATURE: synthetic
QNQELK

SEQ ID NO: 253 - protein, 16 aa, Homo sapiens
  MISC_FEATURE: synthetic
VELLHNPAFCSLATTK

SEQ ID NO: 254 - protein, 16 aa, Homo sapiens
  MISC_FEATURE: synthetic
VELLHNPAFCSLATTK

SEQ ID NO: 255 - protein, 9 aa, Homo sapiens
  MISC_FEATURE: synthetic
TGLQEVEVK

SEQ ID NO: 256 - protein, 13 aa, Homo sapiens
  MISC_FEATURE: synthetic
AAVYHHFISDGVR

SEQ ID NO: 257 - protein, 5 aa, Homo sapiens
  MISC_FEATURE: synthetic
TVAVR

SEQ ID NO: 258 - protein, 6 aa, Homo sapiens
  MISC_FEATURE: synthetic
TLDPER

SEQ ID NO: 259 - protein, 5 aa, Homo sapiens
  MISC_FEATURE: synthetic
SEETK

SEQ ID NO: 260 - protein, 12 aa, Homo sapiens
  MISC_FEATURE: synthetic
ENEGFTVTAEGK

SEQ ID NO: 261 - protein, 14 aa, Homo sapiens
  MISC_FEATURE: synthetic
GQGTLSVVTMYHAK

SEQ ID NO: 262 - protein, 7 aa, Homo sapiens
  MISC_FEATURE: synthetic
DQLTCNK

SEQ ID NO: 263 - protein, 7 aa, Homo sapiens
  MISC_FEATURE: synthetic
DQLTCNK

SEQ ID NO: 264 - protein, 4 aa, Homo sapiens
  MISC_FEATURE: synthetic
FDLK

SEQ ID NO: 265 - protein, 11 aa, Homo sapiens
  MISC_FEATURE: synthetic
VTIKPAPETEK

SEQ ID NO: 266 - protein, 6 aa, Homo sapiens
  MISC_FEATURE: synthetic
RPQDAK

SEQ ID NO: 267 - protein, 10 aa, Homo sapiens
  MISC_FEATURE: synthetic
NTMILEICTR

SEQ ID NO: 268 - protein, 10 aa, Homo sapiens
  MISC_FEATURE: synthetic
NTMILEICTR

SEQ ID NO: 269 - protein, 26 aa, Homo sapiens
  MISC_FEATURE: synthetic
GDQDATMSILDISMMTGFAPDTDDLK

SEQ ID NO: 270 - protein, 8 aa, Homo sapiens
  MISC_FEATURE: synthetic
QLANGVDR

SEQ ID NO: 271 - protein, 4 aa, Homo sapiens
  MISC_FEATURE: synthetic
YISK

SEQ ID NO: 272 - protein, 5 aa, Homo sapiens
  MISC_FEATURE: synthetic
YELDK

SEQ ID NO: 273 - protein, 5 aa, Homo sapiens
  MISC_FEATURE: synthetic
AFSDR

SEQ ID NO: 274 - protein, 9 aa, Homo sapiens
  MISC_FEATURE: synthetic
NTLIIYLDK

SEQ ID NO: 275 - protein, 12 aa, Homo sapiens
  MISC_FEATURE: synthetic
VSHSEDDCLAFK

SEQ ID NO: 276 - protein, 12 aa, Homo sapiens
  MISC_FEATURE: synthetic
VSHSEDDCLAFK

SEQ ID NO: 277 - protein, 16 aa, Homo sapiens
  MISC_FEATURE: synthetic
VHQYFNVELIQPGAVK

SEQ ID NO: 278 - protein, 13 aa, Homo sapiens
  MISC_FEATURE: synthetic
VYAYYNLEESCTR

SEQ ID NO: 279 - protein, 13 aa, Homo sapiens
  MISC_FEATURE: synthetic
VYAYYNLEESCTR

SEQ ID NO: 280 - protein, 6 aa, Homo sapiens
  MISC_FEATURE: synthetic
FYHPEK

SEQ ID NO: 281 - protein, 6 aa, Homo sapiens
  MISC_FEATURE: synthetic
VTLEER

SEQ ID NO: 282 - protein, 11 aa, Homo sapiens
  MISC_FEATURE: synthetic
ACEPGVDYVYK

SEQ ID NO: 283 - protein, 19 aa, Homo sapiens
  MISC_FEATURE: synthetic
VQLSNDFDEYIMAIEQTIK

SEQ ID NO: 284 - protein, 12 aa, Homo sapiens
  MISC_FEATURE: synthetic
SGSDEVQVGQQR

SEQ ID NO: 285 - protein, 7 aa, Homo sapiens
  MISC_FEATURE: synthetic
TFISPIK

SEQ ID NO: 286 - protein, 4 aa, Homo sapiens
  MISC_FEATURE: synthetic
EALK

SEQ ID NO: 287 - protein, 4 aa, Homo sapiens
  MISC_FEATURE: synthetic
LEEK

SEQ ID NO: 288 - protein, 24 aa, Homo sapiens
  MISC_FEATURE: synthetic
HYLMWGLSSDFWGEKPNLSYIIGK

SEQ ID NO: 289 - protein, 20 aa, Homo sapiens
  MISC_FEATURE: synthetic
DTWVEHWPEEDECQDEENQK

SEQ ID NO: 290 - protein, 20 aa, Homo sapiens
  MISC_FEATURE: synthetic
DTWVEHWPEEDECQDEENQK

SEQ ID NO: 291 - protein, 11 aa, Homo sapiens
  MISC_FEATURE: synthetic
ACEPGVDYVYK

(19) United States
(12) Patent Application Publication
(10) Pub. No.: US 2020/0355700 A1
(43) Pub. Date: Nov. 12, 2020

(54) Method for the Diagnosis of Hereditary Angioedema

(71) Applicant: CENTOGENE GMBH, Rostock (DE)

(72) Inventor: Claudia Cozma, Rostock (DE)

(21) Appl. No.: 16/966,652

(22) PCT Filed: Jan. 31, 2019
(86) PCT No.: PCT/EP19/52369

§ 371 Date: Jul. 31, 2020

(30) Foreign Application Priority Data
Jan. 31, 2018 (EP) 18154450.3

(51) Int. Cl. G01N 33/68

(52) U.S. Cl. CPC G01N 33/6893; G01N 2800/224; G01N 33/6848

(57) ABSTRACT

The present invention is related to a method for differential diagnosis of hereditary angioedema, wherein the method comprises determining the level of C4 protein, C1-INH protein and C1q protein in a sample from a subject, wherein the sample is a dried blood spot sample and wherein the level is determined by mass spectrometry.

The present invention is related to a method for differential diagnoses of hereditary angioedema in a subject, and a kit suitable for use in differential diagnoses of hereditary angioedema.

Hereditary angioedema is a rare inherited disorder characterized by recurrent episodes of the accumulation of fluids outside of the blood vessels, blocking the normal flow of blood or lymphatic fluid and causing rapid swelling of tissues in the hands, feet, limbs, face, intestinal tract, or airway. Usually, this swelling is not accompanied by itching, as it might be with an allergic reaction. Swelling of the gastrointestinal tract leads to cramping. Swelling of the airway may lead to obstruction, a potentially very serious complication. These symptoms develop as the result of deficiency or improper functioning of certain proteins that help to maintain the normal flow of fluids through very small blood vessels, i.e. capillaries. In some cases, fluid may accumulate in other internal organs.

The severity of the disease varies greatly among affected individuals. There are three main types of hereditary angioedema, namely Type I, Type II and Type III, with type I being the most common form. Both hereditary angioedema Type I and II are caused by a mutation in the SERPING1 gene that makes the C1 inhibitor protein, which normally suppresses activation of the complement system, while type III is often due to a mutation of the factor XII gene.

Hereditary angioedema is inherited as an autosomal dominant trait. The mutant gene can be inherited from either parent, or can be the result of a spontaneous new mutation in the affected individual.

Treatment of patients with acute attacks includes administration of a plasma derived C1 esterase inhibitor (“Berinert®”, CSL Behring), a kallikrein inhibitor (“Kalbitor®”, Dyax Corporation) or a bradykinin antagonist (“Firazyr®”, Shire). Treatment of patients for long-term prophylaxis include administration of a C1 esterase inhibitor such as “Cinryze®” (Viropharma) and or a 17-α-alkylated androgen such as Danazol (available under the brand names “Danatrol”, “Danocrine”, “Danol”, and “Danoval”)

Hereditary angioedema may be diagnosed by measuring C1-INH levels using either a chromogenic assay or a complex ELISA. Such laboratory tests, i.e. chromogenic assay or a complex ELISA, are nonspecific, time consuming and demand high levels or resources such a biological sample, time and laboratory materials.

The problem underlying the present invention is the provision of a method and means for differential diagnosis of hereditary angioedema.

These and other problems are solved by the subject matter of the attached independent claims. Preferred embodiments may be taken from the attached dependent claims.

More specifically, the problem underlying the present invention is solved in a first aspect by a method for differential diagnosis of hereditary angioedema, wherein the method comprises determining the level of C4 protein, C1-INH protein and C1q protein in a sample from a subject, wherein the sample is a dried blood spot sample and wherein the level is determined by mass spectrometry.

The problem underlying the present invention is solved in a second aspect by a kit suitable for use in a method for differential diagnosis of hereditary angioedema, preferably a method for differential diagnosis of hereditary angioedema according to the first aspect, wherein the kit comprises at least one element selected from the group comprising an interaction partner of one biomarker, one biomarker, instructions of use for the kit, and one or more container, wherein the biomarker is selected from the group comprising C4 protein, a fragment peptide of C4 protein, C1-INH protein, a fragment peptide of C1-INH protein, C1q protein and a fragment peptide of C1q.

The present inventor has surprisingly identified a set of biomarkers which is useful in differently diagnosis of hereditary angioedema. Such set of biomarkers comprises (a) C4 protein or a C4 fragment peptide, (b) C1-INH protein or a C1-INH fragment peptide and (c) C1q protein or a C1q fragment peptide.

In accordance with each and any aspect of the present invention, the biomarker is selected from the group comprising C4 protein, a peptide derived from C4 protein which is, in an embodiment, a C4 fragment peptide, C1-INH protein, a peptide derived from C1-INH protein which is, in an embodiment, a C1-INH fragment peptide, a C1q protein, and a peptide derived from C1q protein which is, in an embodiment, a C1q fragment peptide. It is thus within the present invention that in the practicing of the various methods of the present invention including any aspect and embodiment thereof, the level of C4 protein, C1-INH protein and/or C1q protein is determined. More specifically, it is within the present invention that in the practicing of the various methods of the present invention including any aspect and embodiment thereof, the level of C4 protein, C1-INH protein and C1q protein is determined.

In an embodiment of each and any aspect of the present invention, a peptide derived from C1q protein is a peptide obtained or obtainable upon enzymatic digestion of C1q protein, preferably digestion of C1q protein by tryptic digestion of C1q protein. In an embodiment such peptide is not chemically converted, transformed or derivatized.

Subcomponent C1q of the complement system binds to immunoglobulin complexes with resulting serial activation of C1r (enzyme), C1s (proenzyme), and the other 8 components of complement. C1q is composed of 3 different species of chains, called A, B and C. Preferably any reference herein to C1q or protein C1q refers to both the subcomponent C1q and each and any of its individual chains A, B and C, unless indicated differently.

The amino acid sequence of chain A of C1q is as follows:

(SEQ ID NO: 1)
MEGPRGWLVLCVLAISLASMVTEDLCRAPDGKKGEAGRPGRRGRPGLKG
EQGEPGAPGIRTGIQGLKGDQGEPGPSGNPGKVGYPGPSGPLGARGIPG
IKGTKGSPGNIKDQPRPAFSAIRRNPPMGGNVVIFDTVITNQEEPYQNH
SGRFVCTVPGYYYFTFQVLSQWEICLSIVSSSRGQVRRSLGFCDTTNKG
LFQVVSGGMVLQLQQGDQVWVEKDPKKGHIYQGSEADSVFSGFLIFPSA

The amino acid sequence of chain B of C1q is as follows:

(SEQ ID NO: 2)
MMMKIPWGSIPVLMLLLLLGLIDISQAQLSCTGPPAIPGIPGIPGTPGP
DGQPGTPGIKGEKGLPGLAGDHGEFGEKGDPGIPGNPGKVGPKGPMGPK
GGPGAPGAPGPKGESGDYKATQKIAFSATRTINVPLRRDQTIRFDHVIT
NMNNNYEPRSGKFTCKVPGLYYFTYHASSRGNLCVNLMRGRERAQKVVT
FCDYAYNTFQVTTGGMVLKLEQGENVFLQATDKNSLLGMEGANSIFSGF
LLFPDMEA

The amino acid sequence of chain C of C1q is as follows:

(SEQ ID NO: 3)
MDVGPSSLPHLGLKLLLLLLLLPLRGQANTGCYGIPGMPGLPGAPGKDG
YDGLPGPKGEPGIPAIPGIRGPKGQKGEPGLPGHPGKNGPMGPPGMPGV
PGPMGIPGEPGEEGRYKQKFQSVFTVTRQTHQPPAPNSLIRFNAVLTNP
QGDYDTSTGKFTCKVPGLYYFVYHASHTANLCVLLYRSGVKVVTFCGHT
SKTNQVNSGGVLLRLQVGEEVWLAVNDYYDMVGIQGSDSVFSGFLLFPD

In an embodiment of each and any aspect of the present invention, the fragment peptide derived from C1q is one selected from the following table.

| Peptide | Sequence (N-terminus → C-terminus) |
|---|---|
| C1q-A_[104-110] | GSPGNIK (SEQ ID NO: 4) |
| C1q-A_[111-121] | DQPRPAFSAIR (SEQ ID NO: 5) |
| C1q-A_[123-150] | NPPMGGNVVIFDTVITNQEEPYQNHSGR (SEQ ID NO: |
| | 6) |
| C1q-A_[151-180] | FVCTVPGYYYFTFQVLSQWEICLSIVSSSR (SEQ ID |
| | NO: 7) |
| C1q-A_[151- | FVCTVPGYYYFTFQVLSQWEICLSIVSSSR (SEQ ID |
| 180]_Cys_CAM: 153, 172 | NO: 8) |
| C1q-A_[186-195] | SLGFCDTTNK (SEQ ID NO: 9) |
| C1q-A_[186- | SLGFCDTTNK (SEQ ID NO: 10) |
| 195]_Cys_CAM: 190 | |
| C1q-A_[196-219] | GLFQVVSGGMVLQLQQGDQVWVEK (SEQ ID NO: 11) |
| C1q-A_[224-245] | GHIYQGSEADSVFSGFLIFPSA (SEQ ID NO: 12) |
| C1q-A_[23-27] | EDLCR (SEQ ID NO: 13) |
| C1q-A_[28-32] | APDGK (SEQ ID NO: 14) |
| C1q-A_[34-41] | GEAGRPGR (SEQ ID NO: 15) |
| C1q-A_[49-60] | GEQGEPGAPGIR (SEQ ID NO: 16) |
| C1q-A_[82-94] | VGYPGPSGPLGAR (SEQ ID NO: 17) |
| C1q-B_[118-121] | ATQK (SEQ ID NO: 18) |
| C1q-B_[137-141] | DQTIR (SEQ ID NO: 19) |
| C1q-B_[160-163] | FTCK (SEQ ID NO: 20) |
| C1q-B_[164-177] | VPGLYYFTYHASSR (SEQ ID NO: 21) |
| C1q-B_[178-186] | GNLCVNLMR (SEQ ID NO: 22) |
| C1q-B_[178- | GNLCVNLMR (SEQ ID NO: 23) |
| 186]_Cys_CAM: 181 | |
| C1q-B_[194-215] | VVTFCDYAYNTFQVTTGGMVLK (SEQ ID NO: 24) |
| C1q-B_[194- | VVTFCDYAYNTFQVTTGGMVLK (SEQ ID NO: 25) |
| 215]_Cys_CAM: 198 | |
| C1q-B_[216-229] | LEQGENVFLQATDK (SEQ ID NO: 26) |
| C1q-B_[230-253] | NSLLGMEGANSIFSGFLLFPDMEA (SEQ ID NO: 27) |
| Clq-B_[28-59] | QLSCTGPPAIPGIPGIPGTPGPDGQPGTPGIK (SEQ ID |
| | NO: 28) |
| C1q-B_[28- | QLSCTGPPAIPGIPGIPGTPGPDGQPGTPGIK (SEQ ID |
| 59]_Cys_CAM: 31 | NO: 29) |
| C1q-B_[63-77] | GLPGLAGDHGEFGEK (SEQ ID NO: 30) |
| C1q-B_[78-88] | GDPGIPGNPGK (SEQ ID NO: 31) |
| C1q-B_[93-98] | GPMGPK (SEQ ID NO: 32) |
| C1q-B_[99-110] | GGPGAPGAPGPK (SEQ ID NO: 33) |
| C1q-C_[118-126] | FQSVFTVTR (SEQ ID NO: 34) |
| C1q-C_[127-139] | QTHQPPAPNSLIR (SEQ ID NO: 35) |
| C1q-C_[140-157] | FNAVLTNPQGDYDTSTGK (SEQ ID NO: 36) |
| C1q-C_[162-184] | VPGLYYFVYHASHTANLCVLLYR (SEQ ID NO: 37) |
| C1q-C_[162- | VPGLYYFVYHASHTANLCVLLYR (SEQ ID NO: 38) |
| 184]_Cys_CAM: 179 | |
| C1q-C_[189-198] | VVTFCGHTSK (SEQ ID NO: 39) |
| C1q-C_[189- | VVTFCGHTSK (SEQ ID NO: 40) |
| 198]_Cys_CAM: 193 | |
| C1q-C_[199-210] | TNQVNSGGVLLR (SEQ ID NO: 41) |
| C1q-C_[211-245] | LQVGEEVWLAVNDYYDMVGIQGSDSVFSGFLLFPD |
| | (SEQ ID NO: 42) |
| C1q-C_[29-47] | NTGCYGIPGMPGLPGAPGK (SEQ ID NO: 43) |
| C1q-C_[29- | NTGCYGIPGMPGLPGAPGK (SEQ ID NO: 44) |
| 47]_Cys_CAM: 32 | |
| C1q-C_[48-57] | DGYDGLPGPK (SEQ ID NO: 45) |
| C1q-C_[58-69] | GEPGIPAIPGIR (SEQ ID NO: 46) |
| C1q-C_[76-86] | GEPGLPGHPGK (SEQ ID NO: 47) |
| C1q-C_[87-113] | NGPMGPPGMPGVPGPMGIPGEPGEEGR (SEQ ID NO: |
| | 48) |

A particularly preferred peptide derived from C1q is C1qB_[178-186] which is a compound having a molecular mass m/z of 510.26 as measured with a high resolution ion mobility mass spectrometer and can be measured with MRM-MS, and the amino acid sequence of which is as follows: GNLCVNLMR. This peptide is preferably used as a control and/or for distinguishing between HAE and AAE.

Another particularly preferred peptide derived from C1q is C1qB_[63-77] which is a compound having a molecular mass m/z of 742.36 or 495.24 as measured with a high resolution ion mobility mass spectrometer and can be measured with MRM-MS, and the amino acid sequence of which is as follows: GLPGLAGDHGEFGEK. This peptide is preferably used as a control and/or for distinguishing between HAE and AAE.

In accordance with each and any aspect of the present invention, in an embodiment of such each and any aspect of the present invention, apart from the biomarker the methods comprise the step of determining the presence and/or level of another biomarker, wherein the other biomarker C1-INH protein, a peptide derived from C1-INH, C4 protein and/or a peptide derived from C4 protein.

In an embodiment of each and any aspect of the present invention, a peptide derived from C1-INH protein is a peptide obtained or obtainable upon enzymatic digestion of C1-INH protein, preferably digestion of C1-INH protein by tryptic digestion of C1-INH protein. In an embodiment such peptide is not chemically converted, transformed or derivatized.

In an embodiment of each and any aspect of the present invention, a peptide derived from C4 protein is a peptide obtained or obtainable upon enzymatic digestion of C4 protein, preferably digestion of C4 protein by tryptic digestion of C4 protein. In an embodiment such peptide is not chemically converted, transformed or derivatized.

The amino acid sequence of C1-INH is as follows:

(SEQ ID NO: 49)
MASRLTLLTLLLLLLAGDRASSNPNATSSSSQDPESLQDRGEGKVATTV
ISKMLFVEPILEVSSLPTTNSTTNSATKITANTTDEPTTQPTTEPTTQP
TIQPTQPTTQLPTDSPTQPTTGSFCPGPVTLCSDLESHSTEAVLGDALV
DFSLKLYHAFSAMKKVETNMAFSPFSIASLLTQVLLGAGENTKTNLESI
LSYPKDFTCVHQALKGFTTKGVTSVSQIFHSPDLAIRDTFVNASRTLYS
SSPRVLSNNSDANLELINTWVAKNTNNKISRLLDSLPSDTRLVLLNAIY
LSAKWKTTFDPKKTRMEPFHFKNSVIKVPMMNSKKYPVAHFIDQTLKAK
VGQLQLSHNLSLVILVPQNLKHRLEDMEQALSPSVFKAIMEKLEMSKFQ
PTLLTLPRIKVTTSQDMLSIMEKLEFFDFSYDLNLCGLTEDPDLQVSAM
QHQTVLELTETGVEAAAASAISVARTLLVFEVQQPFLFVLWDQQHKFPV
FMGRVYDPRA

In an embodiment of each and any aspect of the present invention, the fragment peptide derived from C1-INH is one selected from the following table.

| Peptide | Sequence (N-terminus → C-terminus) |
|---|---|
| SerpinG1_[202-211] | DFTCVHQALK (SEQ ID NO: 50) |
| SerpinG1_[202- | DFTCVHQALK (SEQ ID NO: 51) |
| 211]_Cys_CAM: 205 | |
| SerpinG1_[212-216] | GFTTK (SEQ ID NO: 52) |
| SerpinG1_[217-233] | GVTSVSQIFHSPDLAIR (SEQ ID NO: 53) |
| SerpinG1_[23-40] | NPNATSSSSQDPESLQDR (SEQ ID NO: 54) |
| SerpinG1_[234-241] | DTFVNASR (SEQ ID NO: 55) |
| SerpinG1_[242-249] | TLYSSSPR (SEQ ID NO: 56) |
| SerpinG1_[250-268] | VLSNNSDANLELINTWVAK (SEQ ID NO: 57) |
| SerpinG1_[269-273] | NTNNK (SEQ ID NO: 58) |
| SerpinG1_[274-276] | ISR |
| SerpinG1_[277-286] | LLDSLPSDTR (SEQ ID NO: 59) |
| SerpinG1_[301-306] | TTFDPK (SEQ ID NO: 60) |
| SerpinG1_[310-316] | MEPFHFK (SEQ ID NO: 61) |
| SerpinG1_[322-328] | VPMMNSK (SEQ ID NO: 62) |
| SerpinG1_[330-341] | YPVAHFIDQTLK (SEQ ID NO: 63) |
| SerpinG1_[344-364] | VGQLQLSHNLSLVILVPQNLK (SEQ ID NO: 64) |
| SerpinG1_[367-380] | LEDMEQALSPSVFK (SEQ ID NO: 65) |
| SerpinG1_[381-385] | AIMEK (SEQ ID NO: 66) |
| SerpinG1_[386-390] | LEMSK (SEQ ID NO: 67) |
| SerpinG1_[391-400] | FQPTLLTLPR (SEQ ID NO: 68) |
| SerpinG1_[403-415] | VTTSQDMLSIMEK (SEQ ID NO: 69) |
| SerpinG1_[41-44] | GEGK (SEQ ID NO: 70) |
| SerpinG1_[416-466] | LEFFDFSYDLNLCGLTEDPDLQVSAMQHQTVLELTETGV |
| | EAAAASAISVAR (SEQ ID NO: 71) |
| SerpinG1_[467-487] | TLLVFEVQQPFLFVLWDQQHK (SEQ ID NO: 72) |
| SerpinG1_[488-494] | FPVFMGR (SEQ ID NO: 73) |
| SerpinG1_[495-499] | VYDPR (SEQ ID NO: 74) |
| SerpinG1_[53-77] | MLFVEPILEVSSLPTTNSTTNSATK (SEQ ID NO: 75) |

Particularly preferred fragment peptides derived from C1-INH are SerpinG1_[242-249] and SerpinG1_[391-400], whereby SerpinG1_[242-249] with MRM transition 455.74→696.33 is particularly preferred.

The amino acid sequence of C4 is as follows:

(SEQ ID NO: 76)
MRLLWGLIWASSFFTLSLQKPRLLLFSPSVVHLGVPLSVGVQLQ
DVPRGQVVKGSVFLRNPSRNNVPCSPKVDFTLSSERDFALLSLQ
VPLKDAKSCGLHQLLRGPEVQLVAHSPWLKDSLSRTTNIQGINL
LFSSRRGHLFLQTDQPIYNPGQRVRYRVFALDQKMRPSTDTITV
MVENSHGLRVRKKEVYMPSSIFQDDFVIPDISEPGTWKISARFS
DGLESNSSTQFEVKKYVLPNFEVKITPGKPYILTVPGHLDEMQL
DIQARYIYGKPVQGVAYVRFGLLDEDGKKTFFRGLESQTKLVNG
QSHISLSKAEFQDALEKLNMGITDLQGLRLYVAAAIIESPGGEM
EEAELTSWYFVSSPFSLDLSKTKRHLVPGAPFLLQALVREMSGS
PASGIPVKVSATVSSPGSVPEVQDIQQNTDGSGQVSIPIIIPQT
ISELQLSVSAGSPHPAIARLTVAAPPSGGPGFLSIERPDSRPPR
VGDTLNLNLRAVGSGATFSHYYYMILSRGQIVFMNREPKRTLTS
VSVFVDHHLAPSFYFVAFYYHGDHPVANSLRVDVQAGACEGKLE
LSVDGAKQYRNGESVKLHLETDSLALVALGALDTALYAAGSKSH
KPLNMGKVFEAMNSYDLGCGPGGGDSALQVFQAAGLAFSDGDQW
TLSRKRLSCPKEKTTRKKRNVNFQKAINEKLGQYASPTAKRCCQ
DGVTRLPMMRSCEQRAARVQQPDCREPFLSCCQFAESLRKKSRD
KGQAGLQRALEILQEEDLIDEDDIPVRSFFPENWLWRVETVDRF
QILTLWLPDSLTTWEIHGLSLSKTKGLCVATPVQLRVFREFHLH
LRLPMSVRRFEQLELRPVLYNYLDKNLTVSVHVSPVEGLCLAGG
GGLAQQVLVPAGSARPVAFSVVPTAAAAVSLKVVARGSFEFPVG
DAVSKVLQIEKEGAIHREELVYELNPLDHRGRTLEIPGNSDPNM
IPDGDFNSYVRVTASDPLDTLGSEGALSPGGVASLLRLPRGCGE
QTMIYLAPTLAASRYLDKTEQWSTLPPETKDHAVDLIQKGYMRI
QQFRKADGSYAAWLSRDSSTWLTAFVLKVLSLAQEQVGGSPEKL
QETSNWLLSQQQADGSFQDPCPVLDRSMQGGLVGNDETVALTAF
VTIALHHGLAVFQDEGAEPLKQRVEASISKANSFLGEKASAGLL
GAHAAAITAYALTLTKAPVDLLGVAHNNLMAMAQETGDNLYWGS
VTGSQSNAVSPTPAPRNPSDPMPQAPALWIETTAYALLHLLLHE
GKAEMADQASAWLTRQGSFQGGFRSTQDTVIALDALSAYWIASH
TTEERGLNVTLSSTGRNGFKSHALQLNNRQIRGLEEELQFSLGS
KINVKVGGNSKGTLKVLRTYNVLDMKNTTCQDLQIEVTVKGHVE
YTMEANEDYEDYEYDELPAKDDPDAPLQPVTPLQLFEGRRNRRR
REAPKLTSLSDRYVSHFETEGPHVLLYFDSVPTSRECVGFEAVQ
EVPVGLVQPASATLYDYYNPERRCSVFYGAPSKSRLLATLCSAE
VCQCAEGKCPRQRRALERGLQDEDGYRMKFACYYPRVEYGFQVK
VLREDSRAAFRLFETKITQVLHFTKDVKAAANQMRNFLVRASCR
LRLEPGKEYLIMGLDGATYDLEGHPQYLLDSNSWIEEMPSERLC
RSTRQRAACAQLNDFLQEYGTQGCQV

In an embodiment of each and any aspect of the present invention, the fragment peptide derived from C4 is one selected from the following table.

| Peptide | Sequence (N-terminus → C-terminus) |
|---|---|
| C4Alpha_[1006-1008] | LPR |
| C4Alpha_[1009-1026] | GCGEQTMIYLAPTLAASR (SEQ ID NO: 77) |
| C4Alpha_[1009- | GCGEQTMIYLAPTLAASR (SEQ ID NO: 78) |
| 1026]_Cys_CAM: 1010 | |
| C4Alpha_[1027-1030] | YLDK (SEQ ID NO: 79) |
| C4Alpha_[1031-1042] | TEQWSTLPPETK (SEQ ID NO: 80) |
| C4Alpha_[1043-1051] | DHAVDLIQK (SEQ ID NO: 81) |
| C4Alpha_[1052-1055] | GYMR (SEQ ID NO: 82) |
| C4Alpha_[1062-1072] | ADGSYAAWLSR (SEQ ID NO: 83) |
| C4Alpha_[1073-1084] | GSSTWLTAFVLK (SEQ ID NO: 84) |
| C4Alpha_[1085-1099] | VLSLAQEQVGGSPEK (SEQ ID NO: 85) |
| C4Alpha_[1100-1126] | LQETSNWLLSQQQADGSFQDLSPVIHR (SEQ ID NO: |
| | 86) |
| C4Alpha_[1168-1174] | VEASISK (SEQ ID NO: 87) |
| C4Alpha_[1175-1182] | ASSFLGEK (SEQ ID NO: 88) |
| C4Alpha_[1183-1204] | ASAGLLGAHAAAITAYALTLTK (SEQ ID NO: 89) |
| C4Alpha_[1211-1248] | GVAHNNLMAMAQETGDNLYWGSVTGSQSNAVSP |
| | TPAPR (SEQ ID NO: 90) |
| C4Alpha_[1249-1278] | NPSDPMPQAPALWIETTAYALLHLLLHEGK (SEQ |
| | ID NO: 91) |
| C4Alpha_[1279-1291] | AEMADQAAAWLTR (SEQ ID NO: 92) |
| C4Alpha_[1292-1300] | QGSFQGGFR (SEQ ID NO: 93) |
| C4Alpha_[1301-1325] | STQDTVIALDALSAYWIASHTTEER (SEQ ID NO: 94) |
| C4Alpha_[1326-1336] | GLNVTLSSTGR (SEQ ID NO: 95) |
| C4Alpha_[1337-1340] | NGFK (SEQ ID NO: 96) |
| C4Alpha_[1341-1349] | SHALQLNNR (SEQ ID NO: 97) |
| C4Alpha_[1350-1352] | QIR |
| C4Alpha_[1353-1365] | GLEEELQFSLGSK (SEQ ID NO: 98) |
| C4Alpha_[1370-1375] | VGGNSK (SEQ ID NO: 99) |
| C4Alpha_[1383-1390] | TYNVLDMK (SEQ ID NO: 100) |
| C4Alpha_[1391-1404] | NTTCQDLQIEVTVK (SEQ ID NO: 101) |
| C4Alpha_[1391- | NTTCQDLQIEVTVK (SEQ ID NO: 102) |
| 1404]_Cys_CAM: 1394 | |
| C4Alpha_[1405-1428] | GHVEYTMEANEDYEDYEYDELPAK (SEQ ID NO: |
| | 103) |
| C4Alpha_[1429-1446] | DDPDAPLQPVTPLQLFEG (SEQ ID NO: 104) |
| C4Alpha_[680-685] | NVNFQK (SEQ ID NO: 105) |
| C4Alpha_[686-690] | AINEK (SEQ ID NO: 106) |
| C4Alpha_[691-700] | LGQYASPTAK (SEQ ID NO: 107) |
| C4Alpha_[702- | CCQDGVTR (SEQ ID NO: 108) |
| 7091_Cys_CAM: 702, 703 | |
| C4Alpha_[710-714] | LPMMR (SEQ ID NO: 109) |
| C4Alpha_[715- | SCEQR (SEQ ID NO: 110) |
| 719]_Cys_CAM: 716 | |
| C4Alpha_[723-729] | VQQPDCR (SEQ ID NO: 111) |
| C4Alpha_[723- | VQQPDCR (SEQ ID NO: 112) |
| 729]_Cys_CAM: 728 | |
| C4Alpha_[730- | EPFLSCCQFAESLR (SEQ ID NO: 113) |
| 743]_Cys_CAM: 735, 736 | |
| C4Alpha_[750-756] | GQAGLQR (SEQ ID NO: 114) |
| C4Alpha_[757-775] | ALEILQEEDLIDEDDIPVR (SEQ ID NO: 115) |
| C4Alpha_[776-785] | SFFPENWLWR (SEQ ID NO: 116) |
| C4Alpha_[786-791] | VETVDR (SEQ ID NO: 117) |
| C4Alpha_[792-815] | FQILTLWLPDSLTTWEIHGLSLSK (SEQ ID NO: 118) |
| C4Alpha_[818-828] | GLCVATPVQLR (SEQ ID NO: 119) |
| C4Alpha_[818- | GLCVATPVQLR (SEQ ID NO: 120) |
| 828]_Cys_CAM: 820 | |
| C4Alpha_[832-838] | EFHLHLR (SEQ ID NO: 121) |
| C4Alpha_[846-861] | FEQLELRPVLYNYLDK (SEQ ID NO: 122) |
| C4Alpha_[862-912] | NLTVSVHVSPVEGLCLAGGGGLAQQVLVPAGSARP |
| | VAFSVVPTAATAVSLK (SEQ ID NO: 123) |
| C4Alpha_[862- | NLTVSVHVSPVEGLCLAGGGGLAQQVLVPAGSARP |
| 912]_Cys_CAM: 876 | VAFSVVPTAATAVSLK (SEQ ID NO: 124) |
| C4Alpha_[913-916] | VVAR (SEQ ID NO: 125) |
| C4Alpha_[917-929] | GSFEFPVGDAVSK (SEQ ID NO: 126) |
| C4Alpha_[936-941] | EGAIHR (SEQ ID NO: 127) |
| C4Alpha_[942-954] | EELVYELNPLDHR (SEQ ID NO: 128) |
| C4Alpha_[957-979] | TLEIPGNSDPNMIPDGDFNSYVR (SEQ ID NO: 129) |
| C4Alpha_[980-1005 | VTASDPLDTLGSEGALSPGGVASLLR (SEQ ID NO: |
| | 130) |
| C4Beta_[105-118] | GPEVQLVAHSPWLK (SEQ ID NO: 131) |
| C4Beta_[119-123] | DSLSR (SEQ ID NO: 132) |
| C4Beta_[124-137] | TTNIQGINLLFSSR (SEQ ID NO: 133) |
| C4Beta_[139-155] | GHLFLQTDQPIYNPGQR (SEQ ID NO: 134) |
| C4Beta_[158-159] | YR |
| C4Beta_[160-166] | VFALDQK (SEQ ID NO: 135) |
| C4Beta_[167-185] | MRPSTDTITVMVENSHGLR (SEQ ID NO: 136) |
| C4Beta_[190-214] | EVYMPSSIFQDDFVIPDISEPGTWK (SEQ ID NO: 137) |
| C4Beta_[219-234] | FSDGLESNSSTQFEVK (SEQ ID NO: 138) |
| C4Beta_[23-48] | LLLFSPSVVHLGVPLSVGVQLQDVPR (SEQ ID NO: |
| | 139) |
| C4Beta_[236-244] | YVLPNFEVK (SEQ ID NO: 140) |
| C4Beta_[245-269] | ITPGKPYILTVPGHLDEMQLDIQAR (SEQ ID NO: |
| | 141) |
| C4Beta_[270-283] | YIYGKPVQGVAYVR (SEQ ID NO: 142) |
| C4Beta_[284-292] | FGLLDEDGK (SEQ ID NO: 143) |
| C4Beta_[294-297] | TFFR (SEQ ID NO: 144) |
| C4Beta_[298-304] | GLESQTK (SEQ ID NO: 145) |
| C4Beta_[305-316] | LVNGQSHISLSK (SEQ ID NO: 146) |
| C4Beta_[326-337] | LNMGITDLQGLR (SEQ ID NO: 147) |
| C4Beta_[338-373] | LYVAAAIIESPGGEMEEAELTSWYFVSSPFSLDLSK |
| | (SEQ ID NO: 148) |
| C4Beta_[392-404] | EMSGSPASGIPVK (SEQ ID NO: 149) |
| C4Beta_[405-459] | VSATVSSPGSVPEVQDIQQNTDGSGQVSIPIIIPQTISE |
| | LQLSVSAGSPHPAIAR (SEQ ID NO: 150) |
| C4Beta_[460-484] | LTVAAPPSGGPGFLSIERPDSRPPR (SEQ ID NO: 151) |
| C4Beta_[485-494] | VGDTLNLNLR (SEQ ID NO: 152) |
| C4Beta_[49-53] | GQVVK (SEQ ID NO: 153) |
| C4Beta_[495-512] | AVGSGATFSHYYYMILSR (SEQ ID NO: 154) |
| C4Beta_[513-520] | GQIVFMNR (SEQ ID NO: 155) |
| C4Beta_[521-523] | EPK |
| C4Beta_[525-559] | TLTSVSVFVDHHLAPSFYFVAFYYHGDHPVANSLR |
| | (SEQ ID NO: 156) |
| C4Beta_[560-570] | VDVQAGACEGK (SEQ ID NO: 157) |
| C4Beta_[560- | VDVQAGACEGK (SEQ ID NO: 158) |
| 570]_Cys_CAM: 567 | |
| C4Beta_[571-579] | LELSVDGAK (SEQ ID NO: 159) |
| C4Beta_[580-582] | QYR |
| C4Beta_[583-588] | NGESVK (SEQ ID NO: 160) |
| C4Beta_[589-614] | LHLETDSLALVALGALDTALYAAGSK (SEQ ID NO: |
| | 161) |
| C4Beta_[60-63] | NPSR (SEQ ID NO: 162) |
| C4Beta_[615-623] | SHKPLNMGK (SEQ ID NO: 163) |
| C4Beta_[624-664] | VFEAMNSYDLGCGPGGGDSALQVFQAAGLAFSDG |
| | DQWTLSR (SEQ ID NO: 164) |
| C4Beta_[624- | VFEAMNSYDLGCGPGGGDSALQVFQAAGLAFSDG |
| 664]_Cys_CAM: 635 | DQWTLSR (SEQ ID NO: 165) |
| C4Beta_[64-71] | NNVPCSPK (SEQ ID NO: 166) |
| C4Beta_[64- | NNVPCSPK (SEQ ID NO: 167) |
| 71]_Cys_CAM: 68 | |
| C4Beta_[667-671] | LSCPK (SEQ ID NO: 168) |
| C4Beta_[667- | LSCPK (SEQ ID NO: 169) |
| 671]_Cys_CAM: 669 | |
| C4Beta_[72-80] | VDFTLSSER (SEQ ID NO: 170) |
| C4Beta_[81-92] | DFALLSLQVPLK (SEQ ID NO: 171) |
| C4Beta_[93-95] | DAK |
| C4Beta_[96-104] | SCGLHQLLR (SEQ ID NO: 172) |
| C4Beta_[96- | SCGLHQLLR (SEQ ID NO: 173) |
| 104]_Cys_CAM: 97 | |
| C4Gamma_[1458-1465] | VVEEQESR (SEQ ID NO: 174) |
| C4Gamma_[1466-1474] | VHYTVCIVVR (SEQ ID NO: 175) |
| C4Gamma_[1466- | VHYTVCIVVR (SEQ ID NO: 176) |
| 1474]_Cys_CAM: 1471] | |
| C4Gamma_[1475-1477] | NGK |
| C4Gamma_[1478-1498] | VGLSGMAIADVTLLSGFHALR (SEQ ID NO: 177) |
| C4Gamma_[1499-1503] | ADLEK (SEQ ID NO: 178) |
| C4Gamma_[1504-1510] | LTSLSDR (SEQ ID NO: 179) |
| C4Gamma_[1511-1533] | YVSHFETEGPHVLLYFDSVPTSR (SEQ ID NO: 180) |
| C4Gamma_[1534-1564] | ECVGFEAVQEVPVGLVQPASATLYDYYNPER (SEQ |
| | ID NO: 181) |
| C4Gamma_[1534- | ECVGFEAVQEVPVGLVQPASATLYDYYNPER (SEQ |
| 1564]_Cys_CAM: 1535 | ID NO: 182) |
| C4Gamma_[1566-1575] | CSVFYGAPSK (SEQ ID NO: 183) |
| C4Gamma_[1566- | CSVFYGAPSK (SEQ ID NO: 184) |
| 1575]_Cys_CAM: 1566 | |
| C4Gamma_[1578-1594] | LLATLCSAEVCQCAEGK (SEQ ID NO: 185) |
| C4Gamma_[1578- | LLATLCSAEVCQCAEGK (SEQ ID NO: 186) |
| 1594]_Cys_CAM: 1583, | |
| 1588, 1590 | |
| C4Gamma_[1595-1597] | CPR |
| C4Gamma_[1595- | CPR |
| 1597]_Cys_CAM: 1595 | |
| C4Gamma_[1601-1604] | ALER (SEQ ID NO: 188) |
| C4Gamma_[1616-1622] | FACYYPR (SEQ ID NO: 189) |
| C4Gamma_[1616- | FACYYPR (SEQ ID NO: 190) |
| 1622]_Cys_CAM: 1618 | |
| C4Gamma_[1623-1630] | VEYGFQVK (SEQ ID NO: 191) |
| C4Gamma_[1631-1633] | VLR |
| C4Gamma_[1638-1641] | AAFR (SEQ ID NO: 192) |
| C4Gamma_[1642-1646] | LFETK (SEQ ID NO: 193) |
| C4Gamma_[1656-1658] | DVK |
| C4Gamma_[1659-1665] | AAANQMR (SEQ ID NO: 194) |
| C4Gamma_[1671-1674] | ASCR (SEQ ID NO: 195) |
| C4Gamma_[1677-1681] | LEPGK (SEQ ID NO: 196) |
| C4Gamma_[1682-1716] | EYLIMGLDGATYDLEGHPQYLLDSNSWIEEMPSER |
| | (SEQ ID NO: 196) |
| C4Gamma_[1720-1722] | STR |
| C4Gamma_[1725-1744] | AACAQLNDFLQEYGTQGCQV (SEQ ID NO: 197) |
| C4Gamma_[1725- | AACAQLNDFLQEYGTQGCQV (SEQ ID NO: 198) |
| 1744]_Cys_CAM: 1727, | |
| 1742 | |

Particularly preferred fragment peptides derived from C4 are C4Beta[571-579], C4Alpha[680-685], C4Alpha[786-791], C4Beta[294-297], whereby C4Beta[571-579] with MRM transition 466.26→243.13 is particularly preferred.

In connection with each and any aspect of the present invention, C3 protein and/or a peptide derived from C3 protein may be used, preferably as an internal control for the proper functioning of the detection system, preferably of the analysis technique used for determining the level of the biomarker.

A peptide derived from C3 is a peptide obtained or obtainable upon enzymatic digestion of C3 protein, preferably digestion of C3 protein by tryptic digestion of C3 protein.

Component C3 of the complement system plays several important biologic roles in the classical, alternative, and lectin activation pathways, e.g., (1) formation of C3- and C5-convertases, both essential for the full activation of the system; (2) production of opsonins that enhance phagocytosis of microorganisms; (3) degranulation of mast cells and basophils medicated by the fragments C3a and C5a; (4) solubilization and clearance of C3b-bound immune complexes; (5) adjuvant function of fragments C3d and C3dg; and (6) clearance of apoptotic cells. Hereditary angioedema patients typically have normal C3 levels.

The amino acid sequence of C3 is as follows:

(SEQ ID NO: 199)
MGPTSGPSLLLLLLTHLPLALGSPMYSIITPNILRLESEETMV
LEAHDAQGDVPVTVTVHDFPGKKLVLSSEKTVLTPATNHMGNV
TFTIPANREFKSEKGRNKFVTVQATFGTQVVEKVVLVSLQSGY
LFIQTDKTIYTPGSTVLYRIFTVNHKLLPVGRTVMVNIENPEG
IPVKQDSLSSQNQLGVLPLSWDIPELVNMGQWKIRAYYENSPQ
QVFSTEFEVKEYVLPSFEVIVEPTEKFYYIYNEKGLEVTITAR
FLYGKKVEGTAFVIFGIQDGEQRISLPESLKRIPIEDGSGEVV
LSRKVLLDGVQNPRAEDLVGKSLYVSATVILHSGSDMVQAERS
GIPIVTSPYQIHFTKTPKYFKPGMPFDLMVFVTNPDGSPAYRV
PVAVQGEDTVQSLTQGDGVAKLSINTHPSQKPLSITVRTKKQE
LSEAEQATRTMQALPYSTVGNSNNYLHLSVLRTELRPGETLNV
NFLLRMDRAHEAKIRYYTYLIMNKGRLLKAGRQVREPGQDLVV
LPLSITTDFIPSFRLVAYYTLIGASGQREVVADSVWVDVKDSC
VGSLVVKSGQSEDRQPVPGQQMTLKIEGDHGARVVLVAVDKGV
FVLNKKNKLTQSKIWDVVEKADIGCTPGSGKDYAGVFSDAGLT
FTSSSGQQTAQRAELQCPQPAARRRRSVQLTEKRMDKVGKYPK
ELRKCCEDGMRENPMRFSCQRRTRFISLGEACKKVFLDCCNYI
TELRRQHARASHLGLARSNLDEDIIAEENIVSRSEFPESWLWN
VEDLKEPPKNGISTKLMNIFLKDSITTWEILAVSMSDKKGICV
ADPFEVTVMQDFFIDLRLPYSVVRNEQVEIRAVLYNYRQNQEL
KVRVELLHNPAFCSLATTKRRHQQTVTIPPKSSLSVPYVIVPL
KTGLQEVEVKAAVYHHFISDGVRKSLKVVPEGIRMNKTVAVRT
LDPERLGREGVQKEDIPPADLSDQVPDTESETRILLQGTPVAQ
MTEDAVDAERLKHLIVTPSGCGEQNMIGMTPTVIAVHYLDETE
QWEKFGLEKRQGALELIKKGYTQQLAFRQPSSAFAAFVKRAPS
TWLTAYVVKVFSLAVNLIAIDSQVLCGAVKWLILEKQKPDGVF
QEDAPVIHQEMIGGLRNNNEKDMALTAFVLISLQEAKDICEEQ
VNSLPGSITKAGDFLEANYMNLQRSYTVAIAGYALAQMGRLKG
PLLNKFLTTAKDKNRWEDPGKQLYNVEATSYALLALLQLKDFD
FVPPVVRWLNEQRYYGGGYGSTQATFMVFQALAQYQKDAPDHQ
ELNLDVSLQLPSRSSKITHRIHWESASLLRSEETKENEGFTVT
AEGKGQGTLSVVTMYHAKAKDQLTCNKFDLKVTIKPAPETEKR
PQDAKNTMILEICTRYRGDQDATMSILDISMMTGFAPDTDDLK
QLANGVDRYISKYELDKAFSDRNTLIIYLDKVSHSEDDCLAFK
VHQYFNVELIQPGAVKVYAYYNLEESCTRFYHPEKEDGKLNKL
CRDELCRCAEENCFIQKSDDKVTLEERLDKACEPGVDYVYKTR
LVKVQLSNDFDEYIMAIEQTIKSGSDEVQVGQQRTFISPIKCR
EALKLEEKKHYLMWGLSSDFWGEKPNLSYIIGKDTWVEHWPEE
DECQDEENQKQCQDLGAFTESMVVFGCPN

In an embodiment of each and any aspect of the present invention, the fragment peptide derived from C3 is one selected from the following table.

| Peptide | Sequence (N-terminus → C-terminus) |
|---|---|
| C3Beta_[105-119] | FVTVQATFGTQVVEK (SEQ ID NO: 200) |
| C3Beta_[120-136] | VVLVSLQSGYLFIQTDK (SEQ ID NO: 201) |
| C3Beta_[137-148] | TIYTPGSTVLYR (SEQ ID NO: 202) |
| C3Beta_[149-155] | IFTVNHK (SEQ ID NO: 203) |
| C3Beta_[156-161] | LLPVGR (SEQ ID NO: 204) |
| C3Beta_[162-176] | TVMVNIENPEGIPVK (SEQ ID NO: 205) |
| C3Beta_[177-205] | QDSLSSQNQLGVLPLSWDIPELVNMGQWK (SEQ ID |
| | NO: 206) |
| C3Beta_[208-225] | AYYENSPQQVFSTEFEVK (SEQ ID NO: 207) |
| C3Beta_[226-241] | EYVLPSFEVIVEPTEK (SEQ ID NO: 208) |
| C3Beta_[23-35] | SPMYSIITPNILR (SEQ ID NO: 209) |
| C3Beta_[242-249] | FYYIYNEK (SEQ ID NO: 210) |
| C3Beta_[250-258] | GLEVTITAR (SEQ ID NO: 211) |
| C3Beta_[259-263] | FLYGK (SEQ ID NO: 212) |
| C3Beta_[265-281] | VEGTAFVIFGIQDGEQR (SEQ ID NO: 213) |
| C3Beta_[291-304] | IPIEDGSGEVVLSR (SEQ ID NO: 214) |
| C3Beta_[306-315] | VLLDGVQNPR (SEQ ID NO: 215) |
| C3Beta_[316-322] | AEDLVGK (SEQ ID NO: 216) |
| C3Beta_[323-343] | SLYVSATVILHSGSDMVQAER (SEQ ID NO: 217) |
| C3Beta_[344-359] | SGIPIVTSPYQIHFTK (SEQ ID NO: 218) |
| C3Beta_[363-386] | YFKPGMPFDLMVFVTNPDGSPAYR (SEQ ID NO: |
| | 219) |
| C3Beta_[36-65] | LESEETMVLEAHDAQGDVPVTVTVHDFPGK (SEQ |
| | ID NO: 220) |
| C3Beta_[387-408] | VPVAVQGEDTVQSLTQGDGVAK (SEQ ID NO: 221) |
| C3Beta_[409-425] | LSINTHPSQKPLSITVR (SEQ ID NO: 222) |
| C3Beta_[429-439] | QELSEAEQATR (SEQ ID NO: 223) |
| C3Beta_[440-462] | TMQALPYSTVGNSNNYLHLSVLR (SEQ ID NO: 224) |
| C3Beta_[463-478] | TELRPGETLNVNFLLR (SEQ ID NO: 225) |
| C3Beta_[479-481] | MDR |
| C3Beta_[482-486] | AHEAK (SEQ ID NO: 226) |
| C3Beta_[489-497] | YYTYLIMNK (SEQ ID NO: 227) |
| C3Beta_[500-502] | LLK |
| C3Beta_[503-505] | AGR |
| C3Beta_[506-508] | QVR |
| C3Beta_[509-530] | EPGQDLVVLPLSITTDFIPSFR (SEQ ID NO: 228) |
| C3Beta_[531-544] | LVAYYTLIGASGQR (SEQ ID NO: 229) |
| C3Beta_[545-556] | EVVADSVWVDVK (SEQ ID NO: 230) |
| C3Beta_[557-566] | DSCVGSLVVK (SEQ ID NO: 231) |
| C3Beta_[557- | DSCVGSLVVK (SEQ ID NO: 232) |
| 566]_Cys_CAM: 559 | |
| C3Beta_[574-584] | QPVPGQQMTLK (SEQ ID NO: 233) |
| C3Beta_[585-592] | IEGDHGAR (SEQ ID NO: 234) |
| C3Beta_[616-622] | IVVDVVEK (SEQ ID NO: 235) |
| C3Beta_[623-633] | ADIGCTPGSGK (SEQ ID NO: 236) |
| C3Beta_[634-657] | DYAGVFSDAGLTFTSSSGQQTAQR (SEQ ID NO: 237) |
| C3Beta_[658-667] | AELQCPQPAA (SEQ ID NO: 238) |
| C3Beta_[658- | AELQCPQPAA (SEQ ID NO: 239) |
| 667]_Cys_CAM: 662 | |
| C3Beta_[67-73] | LVLSSEK (SEQ ID NO: 240) |
| C3Beta_[74-94] | TVLTPATNHMGNVTFTIPANR (SEQ ID NO: 241) |
| C3Beta_[95-97] | EFK |
| C3Beta_[98-100] | SEK |
| C3cAlpha1_[749-764] | SNLDEDIIAEENIVSR (SEQ ID NO: 242) |
| C3cAlpha1_[765-779] | SEFPESWLWNVEDLK (SEQ ID NO: 243) |
| C3cAlpha1_[780-783] | EPPK (SEQ ID NO: 244) |
| C3cAlpha1_[784-789] | NGISTK (SEQ ID NO: 245) |
| C3cAlpha1_[797-812] | DSITTWEILAVSMSDK (SEQ ID NO: 246) |
| C3cAlpha1_[814-834] | GICVADPFEVTVMQDFFIDLR (SEQ ID NO: 247) |
| C3cAlpha1_[814- | GICVADPFEVTVMQDFFIDLR (SEQ ID NO: 248) |
| 834]_Cys_CAM: 816 | |
| C3cAlpha1_[835-841] | LPYSVVR (SEQ ID NO: 249) |
| C3cAlpha1_[842-848] | NEQVEIR (SEQ ID NO: 250) |
| C3cAlpha1_[849-855] | AVLYNYR (SEQ ID NO: 251) |
| C3cAlpha1_[856-861] | QNQELK (SEQ ID NO: 252) |
| C3cAlpha1_[864-879] | VELLHNPAFCSLATTK (SEQ ID NO: 253) |
| C3cAlpha_[1864- | VELLHNPAFCSLATTK (SEQ ID NO: 254) |
| 879]_Cys_CAM: 873 | |
| C3cAlpha1_[905-913] | TGLQEVEVK (SEQ ID NO: 255) |
| C3cAlpha1_[914-926] | AAVYHHFISDGVR (SEQ ID NO: 256) |
| C3cAlpha1_[938-940] | MNK |
| C3cAlpha1_[941-945] | TVAVR (SEQ ID NO: 257) |
| C3cAlpha1_[946-951] | TLDPER (SEQ ID NO: 258) |
| C3cAlpha1_[952-954] | LGR |
| C3cAlpha2_[1321-1325] | SEETK (SEQ ID NO: 259) |
| C3cAlpha2_[1326-1337] | ENEGFTVTAEGK (SEQ ID NO: 260) |
| C3cAlpha2_[1338-1351] | GQGTLSVVTMYHAK (SEQ ID NO: 261) |
| C3cAlpha2_[1354-1360] | DQLTCNK (SEQ ID NO: 262) |
| C3cAlpha2_[1354- | DQLTCNK (SEQ ID NO: 263) |
| 1360]_Cys_CAM: 1358 | |
| C3cAlpha2_[1361-1364] | FDLK (SEQ ID NO: 264) |
| C3cAlpha2_[1365-1375] | VTIKPAPETEK (SEQ ID NO: 265) |
| C3cAlpha2_[1376-1381] | RPQDAK (SEQ ID NO: 266) |
| C3cAlpha2_[1382-1391] | NTMILEICTR (SEQ ID NO: 267) |
| C3cAlpha2_[1382- | NTMILEICTR (SEQ ID NO: 268) |
| 1391]_Cys_CAM: 1389 | |
| C3cAlpha2_[1394-1419] | GDQDATMSILDISMMTGFAPDTDDLK (SEQ ID NO: |
| | 269) |
| C3cAlpha2_[1420-1427] | QLANGVDR (SEQ ID NO: 270) |
| C3cAlpha2_[1428-1431] | YISK (SEQ ID NO: 271) |
| C3cAlpha2_[1432-1436] | YELDK (SEQ ID NO: 272) |
| C3cAlpha2_[1437-1441] | AFSDR (SEQ ID NO: 273) |
| C3cAlpha2_[1442-1450] | NTLIIYLDK (SEQ ID NO: 274) |
| C3cAlpha2_[1451-1462] | VSHSEDDCLAFK (SEQ ID NO: 275) |
| C3cAlpha2_[1451- | VSHSEDDCLAFK (SEQ ID NO: 276) |
| 1462]_Cys_CAM: 1458 | |
| C3cAlpha2_[1463-1478] | VHQYFNVELIQPGAVK (SEQ ID NO: 277) |
| C3cAlpha2_[1479-1491] | VYAYYNLEESCTR (SEQ ID NO: 278) |
| C3cAlpha2_[1479- | VYAYYNLEESCTR (SEQ ID NO: 279) |
| 1491]_Cys_CAM: 1489 | |
| C3cAlpha2_[1492-1497] | FYHPEK (SEQ ID NO: 280) |
| C3cAlpha2_[1502-1504] | LNK |
| C3cAlpha2_[1505-1507] | LCR |
| C3cAlpha2_[1505- | LCR |
| 1507]_Cys_CAM: 1506 | |
| C3cAlpha2_[1527-1532] | VTLEER (SEQ ID NO: 281) |
| C3cAlpha2_[1533-1535] | LDK |
| C3cAlpha2_[1536-1546] | ACEPGVDYVYK (SEQ ID NO: 282) |
| C3cAlpha2_[1536- | ACEPGVDYVYK (SEQ ID NO: 291) |
| 1546]_Cys_CAM: 1537 | |
| C3cAlpha2_[1552-1570] | VQLSNDFDEYIMAIEQTIK (SEQ ID NO: 283) |
| C3cAlpha2_[1571-1582] | SGSDEVQVGQQR (SEQ ID NO: 284) |
| C3cAlpha2_[1583-1589] | TFISPIK (SEQ ID NO: 285) |
| C3cAlpha2_[1592-1595] | EALK (SEQ ID NO: 286) |
| C3cAlpha2_[1596-1599] | LEEK (SEQ ID NO: 287) |
| C3cAlpha2_[1601-1624] | HYLMWGLSSDFWGEKPNLSYIIGK (SEQ ID NO: 288) |
| C3cAlpha2_[1625-1644] | DTWVEHWPEEDECQDEENQK (SEQ ID NO: 289) |
| C3cAlpha2_[1625- | DTWVEHWPEEDECQDEENQK (SEQ ID NO: 290) |
| 1644]_Cys_CAM: 1637 | |

A particularly preferred peptide derived from C3 is C3Beta_[489-497] which is a compound having a molecular mass m/z of 604.81 as measured with a high resolution ion mobility mass spectrometer, MRM transition 604.8→327.22 and which can be measured with MRM-MS, and the amino acid sequence of which is as follows: YYTYLIMNK. Another preferred fragment peptide of C3 is C3cAlpha1_[814-834]_Cys_CAM: 816 with MRM transition 824.74→798.44.

Another particularly preferred peptide derived from C3 is C3cAlpha_[814-834]Cys_CAM816 which is a having a molecular mass m/z of 495.25 as measured with a high resolution ion mobility mass spectrometer and can be measured with MRM-MS, and the amino acid sequence of which is as follows: GICVADPFEVTVMQDFFIDLR. “CAM” refers to carbamidomethyl and is the result of the alkylation of the free SH-groups after cleavage of C3 into peptides.

As preferably used herein, a fragment peptide is a peptide of a protein generated by digestion, preferably complete digestion of the protein by a proteolytic enzyme.

It will be acknowledged that rather than using trypsin for the generation of a peptide from proteins C1q, C1-INH, C4 and C3 respectively, another proteolytic enzyme may be used, preferably the proteolytic enzyme is a protease or peptidase which, upon complete digestion of the protein, provides a mixture of peptides, wherein each species of the peptide is present only once. This ensures that there is a 1:1 stoichiometry between the protein and each and any peptide obtained by such complete digestion of the protein using the protease. In another embodiment, digestion reaction or protease is selected from the group comprising Arg-C, Asp-N, Asp-N(N-terminal Glu), BNPS or NCS/urea, Caspase-1, Caspase-10, Caspase-2, Caspase-3, Caspase-4, Caspase-5, Caspase-6, Caspase-7, Caspase-8, Caspase-9, Chymotrypsin, Chymotrypsin (low specificity), Clostripain, CNBr, CNBr (methyl-Cys), CNBr (with acids), Enterokinase, Factor Xa, Formic acid, Glu-C (AmAc buffer, Glu-C (Phos buffer), Granzyme B, HRV3C protease, Hydroxylamine, Iodosobenzoic acid, Lys-C, Lys-N, Lys-N(Cys modified), Mild acid hydrolysis, NBS (long exposure), NBS (short exposure), NTCB, Pancreatic elastase, Pepsin A, Pepsin A (low specificity), Prolyl endopeptidase, Proteinase K, TEV protease, Thermolysin, Thrombin

It will be further acknowledged by a person skilled in the art that although, in principle, all of the protein derived peptides are suitable for use in any method of any aspect of the present invention, the use of different peptides may be preferred depending on the technique used for the detection of the biomarker. Accordingly, in an embodiment of each any aspect of the invention, the biomarker is a peptide derived from any of proteins C1q, C1-INH and C4 which is particularly suitable for detection by means of mass spectrometry, particularly in case detection is made by mass spectrometry. Also accordingly, in an embodiment of each any aspect of the invention, the biomarker is a peptide derived from any of proteins C1q, C1-INH and C4 against which an antibody or a functional nucleic acid may be generated with the antibody and functional nucleic acid providing for a highly specific and/or highly selective detection and/or quantification of said protein, particularly in case detection is made by means of assay using such antibody or functional nucleic acid as an interaction partner of said peptide.

The term “hereditary angioedema” (HAE), to which it is also referred herein as “the disease”, is a rare inherited disorder characterized by recurrent episodes of the accumulation of fluids outside of the blood vessels, blocking the normal flow of blood or lymphatic fluid and causing rapid swelling of tissues in the hands, feet, eyelids, lips, limbs, face, intestinal tract, airways and genitals. Usually, this swelling is not accompanied by itching, as it might be with an allergic reaction. Swelling of the gastrointestinal tract leads to cramping. Swelling of the airway may lead to obstruction, a potentially very serious complication. These symptoms develop as the result of deficiency or improper functioning of certain proteins that help to maintain the normal flow of fluids through very small blood vessels (capillaries).

In some cases, fluid may accumulate in other internal organs. The severity of the disease varies greatly among affected individuals. Edema may also occur in the mucous membranes that line the respiratory and digestive tracts, which is more common in people with hereditary angioedema than in those who have other forms of angioedema (i.e., acquired or traumatic). People with this disorder typically have areas of swelling that are hard and painful, not red and itchy (pruritic). A skin rash (urticaria) is rarely present.

The symptoms of hereditary angioedema may recur and can become more severe. Injury, severe pain, surgery, dental procedures, viral illness, and/or stress can trigger or worsen the recurring symptoms.

Symptoms associated with swelling in the digestive system (gastrointestinal tract) include nausea, vomiting, acute abdominal pain, and/or other signs of obstruction. Edema of the throat (pharynx) or voice-box (larynx) can result in pain, difficulty swallowing (dysphagia), difficulty speaking (dysphonia), noisy respiration (stridor), and potentially life-threatening asphyxiation.

There are three forms of hereditary angioedema, namely hereditary angioedema type I, hereditary angioedema type II and hereditary angioedema type III.

The most common form of the disorder is hereditary angioedema type I, which is the result of a deficiency of a protein known as complement component C1 esterase inhibitor. In hereditary angioedema type I, representing 85% of patients, serum levels of the C1 esterase inhibitor are less than 35% of normal. In type II, the levels are normal or elevated, but the protein is nonfunctional. The two types are clinically indistinguishable. Hereditary angioedema type III is caused by mutation in the gene encoding coagulation factor XII (F12; 610619) on chromosome 5q.

Hereditary angioedema is inherited as an autosomal dominant trait. The genetic defect underlying hereditary angioedema is a heterozygous mutation in the C1 esterase inhibitor gene (C1NH, SERPING1) on chromosome 11q. Patients with of hereditary angioedema type I appear to have a deletion of the C1 esterase inhibitor gene or a truncated transcript because of a stop codon, whereas patients with of hereditary angioedema type II have a single base substitution. The two forms are clinically indistinguishable. Mutations in the C1 esterase inhibitor gene associated with hereditary angioedema and of mutations in C1 esterase inhibitor gene tested in the diagnosis of hereditary angioedema are known to the person skilled in the art and can be retrieved from scientific papers using routine measures. Mutations in the C1 esterase inhibitor protein associated with hereditary angioedema and of mutations in C1 esterase inhibitor protein tested in the diagnosis of hereditary angioedema are known to the person skilled in the art and can be retrieved from scientific papers using routine measures. Known DNA changes in the C1 esterase inhibitor gene are c.550G>A, c.671T>A, c.551_685 de1, c.−191_51 de1/de1 of exon 1 and 2, c.1081C>T, c.106_107 de1 and c.1397G>A.

In an embodiment of each and any aspect of the present invention, hereditary angioedema is hereditary angioedema type I.

The term “sample” as used herein means preferably a limited quantity of a subject's material, wherein said subject's material is part of or has been taken from a subject and/or a subject's body. Preferably, said material is selected from the group comprising body fluids such as blood, a blood product, urine, saliva, cerebrospinal fluid and lymph, as well as stool or any kind of tissue and or cell material being part of a subject and/or a subject's body. It will be acknowledged by a person skilled in the art that the presence of and/or a level of the biomarker of the invention in said sample is intended to be similar to and represent the presence and/or the level of the biomarker in a larger amount of that subject's material. More precisely and as an illustrative, non-limiting example, a level of the biomarker of the invention determined in a sample of, e.g., some ml of blood from a subject also represents a level of said biomarker in the blood of the subject's body. Furthermore, in an embodiment of the methods of each and any aspect of the invention, a sample from the subject comprises said subject's material in a form, for example processed, fixed and/or preserved such that said sample is suitable for use in the methods of each and any aspect of the invention, whereby such processing, fixing and/or preserving preferably does neither generate the biomarker, at least not unintentionally, which was not as such present in the blood of the patient. The subject's material in the sample may thus be diluted, for example with a solvent suitable for the method of each and any aspect of the invention such as methanol and/or water, may be dried, for example on a filter card, may be resolved after having been dried such, for example with a solvent suitable for the method of the invention such as methanol and/or water, or a substance may be added, wherein said substance prevents blood from coagulation such as for example EDTA or heparin.

A sample as preferably used in connection with each and any aspect of the present invention a sample as used in such methods is prepared from a primary source such as whole blood. Other samples include, but are not limited to serum samples and plasma samples.

In an embodiment of the various aspects of the invention the primary sample is whole blood which is, in an embodiment, processed such that it is collected on a dry blood filter card; preferably approximately 3 μl of full blood are collected on a spot of said dry blood filter card having a diameter of 3 mm. A person skilled in the art will acknowledge that the exact volume thus collected may vary depending on the hematocrit of the specific patient.

In an embodiment of each and any aspect of the present invention where the sample is blood or dry blood spots or other liquids or tissues and wherein the biomarker is a peptide derived from C4 protein, C1q protein and/or from C1-INH protein, the sample may be processed as follows:
extracting of blood components;
subjecting the extract in situ to a reaction with reducing agent, preferably dithiothreitol (DDT), to reduce the disulfide bridges in the proteins and to an alkylation agent, preferably iodacetamide (IAA), to alkylate the free —SH groups;
digesting the mixture into peptides, preferably by use of a protease, more preferably by the use of the protease trypsin; and analyzing the mixture containing peptide fragments of the proteins by mass spectrometry, preferably LC-mass spectrometry analysis, and more preferably in the presence of an internal standard.

In an embodiment of each and any aspect of the method of the invention wherein an internal standard is added to a or the sample, the internal standard may be added to the sample before or after the trypsin digestion step, i.e. the internal standard may be added into the sample immediately after the sample is taken from the subject, or may be added to the supernatant which is subjected to HPLC, as well as in between these points in time. It is within the skills of a person of the art to determine how and when an internal standard is to be added to the sample in order to achieve an accurate detection and determination of a level of the biomarker, wherein according to the present invention preferably the internal standard is added to a sample that contains the biomarker.

It will be acknowledged by a person skilled in the art that by said addition of internal standard, also referred to herein as IS, to the sample, i.e. spiking of the sample, to be subjected to such method according to the present invention, the concentration of IS in the sample is known and, e.g., by determining the area under the peak, i.e. the peak area, of the internal standard in, e.g., an HPLC-mass spectrometric chromatogram the relation between a peak area and a concentration of a substance, e.g. of IS, and/or the biomarker of the present invention is established and thus a means provided for determining the level of the biomarker in the sample. A person skilled in the art will further acknowledge that various molecules may be used as an IS. Nevertheless, an IS having a similar chemical structure compared to the molecule such as the biomarker is preferred. In a preferred embodiment, the molecule being the IS can be distinguished from the biomarker of the present invention. The latter applies in particular to those embodiments of each and any aspect of the present invention where the biomarker is a peptide derived C4 protein, C1-INH protein and/or C1q protein. In a further preferred embodiment of each and any aspect of the present invention, the IS is selected such that a molecule which is ideally not present or rare in nature, is bearing heavy isotopes (such as C13, N15 versions of the biomarker), comprising modified amino acids such as D-amino acids or + or − amino acids, or dextro peptides. In a preferred embodiment of each and any aspect of the present invention Leucine-Enkephaline is used as an internal standard which is not present as such in nature.

In an embodiment of the various aspects of the present invention where the internal standard is added to a sample from a subject, it is preferred that the IS is added such that it is dissolved in a solvent, e.g. water, prior to said addition to the sample.

According to the present invention, including any aspect and embodiment thereof, a biomarker is detected.

As preferably used herein, the term “detecting” means methods which include detecting the presence or absence of a substance in a sample and/or qualifying the type of said substance. In an embodiment the substance is a biomarker, a control and/or an internal standard. Detecting can be accomplished by methods known in the art and those further described herein. These methods include, without limitation, mass spectrometric analysis, biochip array, functional nucleic acids and/immunoassay. Preferably, the biomarker is detected and/or quantified by means of mass-spectrometric analysis. In a more preferred embodiment, mass spectrometric analysis is selected from the group comprising SELDI MS, MALDI MS, ESI MS, DESI MS and ion mobility MS. In an embodiment, mass spectrometric analysis uses an analyzer selected from the group comprising ToF, QToF, ion trap, Triple Quad, orbitrap, FT-ICR, ion mobility and any combination thereof. In an embodiment of the present invention, including any aspect and embodiment thereof, the level of the biomarker is determined by means of mass spectrometric analysis following HPLC separation.

In another embodiment of each and any aspect of the present invention, the biomarker is detected by means of an interaction partner. Such interaction partner is one selected from the group comprising an antibody, an anticaline and a functional nucleic acid. It is within the skills of a person of the art to generate an antibody binding to the biomarker. Antibodies may be generated as known to the one skilled in the art and described, e. g. by Harlow, E., and Lane, D., “Antibodies: A Laboratory Manual,” Cold Spring Harbor Laboratory, Cold Spring Harbor, N.Y., (1988). It is within the skills of a person of the art to generate an anticaline binding to the biomarker. The generation of anticlines is, for example, described in German patent application DE 197 42 706. In an embodiment, the functional nucleic acid is an aptamer. It is within the skills of a person of the art the generate an aptamer. Aptamers are D-nucleic acids which are either single stranded or double stranded and which specifically interact with a target molecule. The generation of aptamers is, for example, described in European patent EP 0 533 838. In an embodiment, the functional nucleic acid is a spiegelmer. It is within the skills of a person of the art the generate a spiegelmer. Spiegelmers are L-nucleic acids which are either single stranded or double stranded and which specifically interact with a target molecule. The generation of aptamers is, for example, described in international patent application WO 98/08856.

It will be understood by a person skilled in the art that the above indicated techniques and methods for detecting the biomarker may be equally used for quantifying the biomarker.

In an embodiment of the present invention, including any aspect and embodiment thereof, the “level” or “level of a biomarker” as preferably used herein, means the concentration or concentration of a biomarker, preferably in a sample of a subject. The level may be an absolute level, expressed, for example, in ng/ml (ng of the compound and biomarker, respectively, in ml of a/the sample). The level may be a relative level. Such relative level is, in an embodiment, the ratio of a/the biomarker to an internal standard. In an embodiment of the present invention, including any aspect and embodiments thereof, is determined as follows, preferably after cleavage of protein C4, C1q and/or C1-INH peptide fragments, and more preferably after alkylation of the free SH-groups of the peptide. In the analytical set-up as described in the example part in more detail, an internal standard is added to the sample to be analyzed. In the course of such analysis a chromatogram is obtained indicating as individual peaks the various compounds detected in the sample. The various compounds include, among others, a fragment of C4 protein, C1q and/or C1-INH protein and the internal standard. In order to determine from such chromatogram and the peaks indicated therein, the concentration or level of a/the fragment peptide(s) the peak area of the peak corresponding to a/the peptide fragment(s) and the peak area of the peak corresponding to the internal standard is determined. Based on the peak area of the fragment peptide(s) and the peak area of the internal standard the ratio of the fragment peptide(s) to the internal standard can be determined. The concentration of a/the fragment peptide(s) is obtained using a standard curve of a/the fragment peptides at different concentrations in the presence of internal standard at known concentration(s).

In embodiment of the present invention, including any aspect and embodiment thereof, the level of a/the biomarker is compared to a level of the same or another biomarker of the present invention determined in another sample, e.g. from the same patient, from another patient, from a control and/or from the same or different points in time, and/or a level of a control and/or a level of an IS. In connection therewith “comparing” or “compared to” as used herein, preferably means the mathematical comparison of the two or more values of the levels or ratios of the biomarker(s). It will thus be immediately evident whether one of said values is higher, lower or identical if at least two of such values or ratios are compared with each other. In an embodiment, such comparison may be carried out using a/the absolute level. In an alternative embodiment, such comparison may be carried out using a/the relative level.

In an embodiment of the present invention, including any aspect and embodiments thereof, the level of the biomarker is also determined in a control. As used herein, a control is preferably a sample from a subject, wherein the hereditary angioedema status of said subject is known. In an embodiment a control is a sample of a healthy patient. In a further embodiment an amount of said biomarker is added to said sample of a healthy patient prior to determining the level of said biomarker in said sample of a healthy patient comprising said added biomarker, preferably in the practicing of a method of the present invention. In a further embodiment the control is a sample from at least one subject having a known hereditary angioedema status, e.g. a control patient, and in a still further preferred embodiment also comprises the genetic status with regard to mutations of the gene, affected in said disease, comprising C1 esterase inhibitor protein, i.e. comprising the subject having homozygous and/or compound heterozygous mutations, the subject being a carrier of a mutation. In a further preferred embodiment, the control is a sample from a subject not being treated for the disease. In a still further preferred embodiment the control is a sample from a single subject or a pool of samples from different subjects and/or samples taken from the subject(s) at different points in time.

In an embodiment of the present invention, including any aspect and embodiments thereof, a subject is considered to be a healthy subject with regard to the disease, if the subject does not suffer from symptoms associated with such disease. More specifically and in an embodiment of the present invention, including any aspect and embodiment thereof, a subject will be considered to be healthy regarding hereditary angioedema, if it has no mutation of the functional parts of the C1 esterase inhibitor gene resulting in a reduction of or deficiency of the respective protein or the activity thereof, resulting in symptoms associated with hereditary angioedema.

In connection with the present invention, including any aspect and embodiments thereof, a “patient” is a subject showing at least one symptom of the disease. More preferably, a patient is a subject presenting one homozygous mutation or multiple heterozygous mutations of the C1 esterase inhibitor gene resulting in reduction or deficiency of the respective protein and/or protein activity, resulting in symptoms associated with hereditary angioedema. Furthermore, in connection therewith a “carrier” is a subject presenting one heterozygous mutation of the C1 esterase inhibitor gene resulting or not resulting in reduction or deficiency of the respective protein and/or protein activity, usually or preferably not resulting in symptoms associated with hereditary angioedema.

In embodiment of the present invention, including any aspect and embodiment thereof, the level of a/the biomarker is compared to a cut-off (which term is synonymously used to the terms cut-off value or cut-off level). The term “cut-off value” as preferably used herein is a level (or concentration) which may be an absolute level or a relative level, which is indicative whether a person is suffering from a disease and/or is at risk of suffering from a disease. Depending on the biomarker, a subject is regarded as suffering the from the diseases or being at risk of suffering from the diseases if either the level of the biomarker detected and determined, respectively, is lower than the cut-off value, or the level of the biomarker detected and determined, respectively, is higher than the cut-off value. As preferably used herein, the cut-off value is set at the mean value of a cohort of healthy subject ±2× standard deviation.

The cut-off value for some of the fragment peptides used in the method for differential diagnosis of hereditary angioedema is as follows.

| Peptide | Cut-off |
|---|---|
| C4Beta_[571 -579] | 500 ng/mL |
| SerpinG1_[242-249] | 835 ng/mL |
| C1q-Beta_[178-186] | 800 ng/mL |
| C4Alpha_[680-685] | 260 ng/mL |
| C4Alpha_[786-791] | 100 ng/mL |
| C4Beta_[294-297] | 201 ng/mL |
| C4Gamma_[1638-1641] | 920 ng/mL |
| SerpinG1_[391-400] | 392 ng/mL |
| C1q-Beta_[63-77] | 1690 ng/mL |

It will be understood by a person skilled in the art that based on the above cut-off values, corresponding cut-off values may be calculated for any of the other fragment peptides based on the molecular weight of the above fragment peptides and said other fragment peptides. The same also applies to the cut-off value of any one of the C4 protein, C1-INH protein and C1q protein and the individual polypeptides forming the same. The cut-off values calculated in such way are also referred to herein as corresponding cut-off values, whereby, preferably reference is made to one or more of the above cut-off values for the indicated fragment peptides.

A “limit of detection” of a substance such as a biomarker of control, as preferably used herein, is a level of the substance determined by a method for determining a level of the substance, wherein a level less then or lower then said limit of detection cannot be determined by said method. It is thus immediately clear that a “cut-off value” and a “limit of detection”, as used herein, are preferably not necessarily identical, although both reflect a certain level of a substance, e.g. of a biomarker of the present invention. Also, it will be immediately understood that a cut-off value will be selected preferably such that selectivity and sensitivity of the method are as high as possible. In contrast thereto, a limit of detection represents an absolute level of the biomarker of the present invention which reflects the minimum level of biomarker which can be detected with a method for determining the level of said biomarker. It is thus immediately clear that a limit of detection depends on the method for determining a level of a substance and on the substance the level of which is to be determined by the method. A skilled person will immediately understand that a high limit of detection, e.g. higher than an ideal cut-off value would possibly result in a low sensitivity of the method since the percentage of true positives that are predicted by a test to be positive also depends on whether a level of the biomarker may be determined for said true positives. In other words, if the limit of detection is higher than an ideal cut-off value, true positives having a level of the biomarker slightly higher than the cut-off value may not be distinguished from true negatives having a level of the biomarker lower than the cut-off value since no level of the biomarker may be determined for both true positives having a level of the biomarker slightly higher than the cut-off value and negatives having a level of the biomarker lower than the cut-off value. It is thus immediately clear that a low limit of detection is of advantage. Preferably, an “ideal cut-off value” as used herein is a cut-off value that has the highest selectivity and sensitivity.

It is within the present invention that the method for diagnosing the disease as subject to the first aspect of the present invention, in one embodiment, encompasses that the subject from whom the sample has been taken, is a subject from whom a sample had been subjected to said method earlier. In a preferred embodiment the time difference between said two samples is 2 weeks, one month, two months or three months; preferably the time difference between said two samples is one month. In accordance therewith, the method of the first aspect, including any embodiment thereof, comprises determining the level of a/the biomarker in a sample from as subject and as a further step determining the level of a/the biomarker in a second sample from the subject, wherein the second sample has been taken from the subject after said time difference.

It is within the present invention that the method for diagnosing the disease as subject to the first aspect of the present invention, in one embodiment, uses a sample taken from a subject to whom a therapy had been applied prior to the point in time when the sample was taken or to whom a therapy was applied at the point in time when the sample was taken.

It is within the present invention that the method for diagnosing the disease as subject to the first aspect of the present invention, in one embodiment, uses a sample taken from a subject to whom no therapy had been applied prior to the point in time when the sample was taken or to whom no therapy was applied at the point in time when the sample was taken.

In a second aspect, the present invention is related to a kit, wherein the kit comprises at least one element selected from the group comprising an interaction partner of a or the biomarker, a or the biomarker, instructions of use for the kit, and one or more containers. In an embodiment, the kit is for use in a method according to the first aspect of the present invention. In a preferred embodiment, the kit comprises an interaction partner of a or the biomarker, preferably an interaction partner for one fragment peptide of each of C4, C1q and C1-IHN or an interaction part for each of C4, C1q and C1-IHN, and instructions for use and, optionally, one or more containers. In another preferred embodiment, the kit comprises a or the biomarker, preferably an interaction partner for one fragment peptide of each of C4, C1q and C1-IHN or an interaction part for each of C4, C1q and C1-IHN, and instructions for use and, optionally, one or more containers.

In an embodiment of the second aspect, the interaction partner is one selected from the group comprising an antibody, an anticaline, an aptamer and a spiegelmer, wherein any one of the antibody, anticaline, aptamer and spiegelmer and spiegelmer is capable of binding to a or the biomarker, preferably the binding is such that a complex is formed between the biomarker and the interaction partner which allows detection and, respectively, quantification of the complex or the biomarker, preferably after dissolution of the complex.

The term “being at risk for developing a disease” as used herein preferably means that it is likely that a subject will suffer from said disease and/or will develop said disease or symptoms associated with said disease, particularly if no treatment is applied. In connection therewith, it has to be acknowledged that hereditary angioedema is a genetic disorder and thus the occurrence of relatives, particularly parents having said disease or having a mutation known to be the cause of said disease are indicative for a subject, e.g. the child of two hereditary angioedema patients or two hereditary angioedema carriers, to be at risk for developing said disease. It will furthermore be acknowledged that the progression of a disease is linked to the occurrence of symptoms as well as the severity of said symptoms. Accordingly, a person not suffering from symptoms at present, however, may be at risk for developing the disease, for example, because although genetically mutations of a gene, known to cause a disease are present, no symptoms or no severe symptoms occur. Nevertheless, it will be immediately understood that the methods and biomarkers of the present invention, particularly if the level of said biomarker according to the present invention is reduced or increased, depending on the biomarker, allow for diagnosing that such subject is at risk for developing the disease independent from the presence or absence of symptoms. Accordingly, the methods according to the present invention allow for determining whether a subject is at risk of suffering from the disease. It is also within the present invention that a therapy is applied, maintained, reduced, elevated or not applied based on whether the subject is at risk of suffering from the disease or not.

The term “qualifying hereditary angioedema status” in a subject as used herein, preferably means a classification of a subject's biomarker profile selected from the group comprising to identify or detect the presence or absence of hereditary angioedema in the subject, to predict the onset of or the risk for developing of hereditary angioedema in the subject, to determine the course of hereditary angioedema in a subject, to determine whether a subject suffers from an early status of hereditary angioedema or an advanced or progressed status of hereditary angioedema or to determine whether a level of a biomarker in a subject has significantly changed over time.

The term “managing subject treatment” or “subject management” as used herein, preferably refers to the behavior of the clinician or physician subsequent to the determination of hereditary angioedema status. For example, if the result of the methods according to the present invention is inconclusive or there is reason that confirmation of status is necessary, the physician may order new tests, such as testing for the function of the affected proteins and/or sequencing of the C1 esterase inhibitor gene. Alternatively, if the status indicates that treating for hereditary angioedema is appropriate, the physician may schedule the subject for treating for hereditary angioedema. Likewise, if the status is negative or if the results show that treatment has been successful, no further management may be necessary. Nevertheless, a person skilled in the art will immediately acknowledge that besides gene therapy any suitable and/or effective therapy may be applied, including the therapy discloses herein. Furthermore, it is an embodiment of the present invention that managing subject treatment comprises titrating of a dose of a drug applied as a treatment for hereditary angioedema, e.g. amount of an C1 esterase inhibitor, a kallikrein inhibitor or a bradykinin antagonist, applied or administered to a patient and/or subject. In some embodiments of the methods of the present invention wherein a level of a biomarker present in a sample from a subject is determined at several points in time, or is compared to other levels of the biomarker, a cut-off value and/or a level of said biomarker in a control and/or another value of a ratio of the levels of two biomarkers, a skilled person will apply or not apply a therapy, or amend a therapy already applied in order to treat or not to treat, or to continue treating hereditary angioedema.

In an embodiment of the present invention, the terms “being at risk of developing the disease” and “being at risk of suffering from the disease” are used interchangeably herein, unless indicated to the contrary.

The present invention is now further illustrated by the following figures and examples from which further features, embodiments and advantages may be taken.

More specifically,

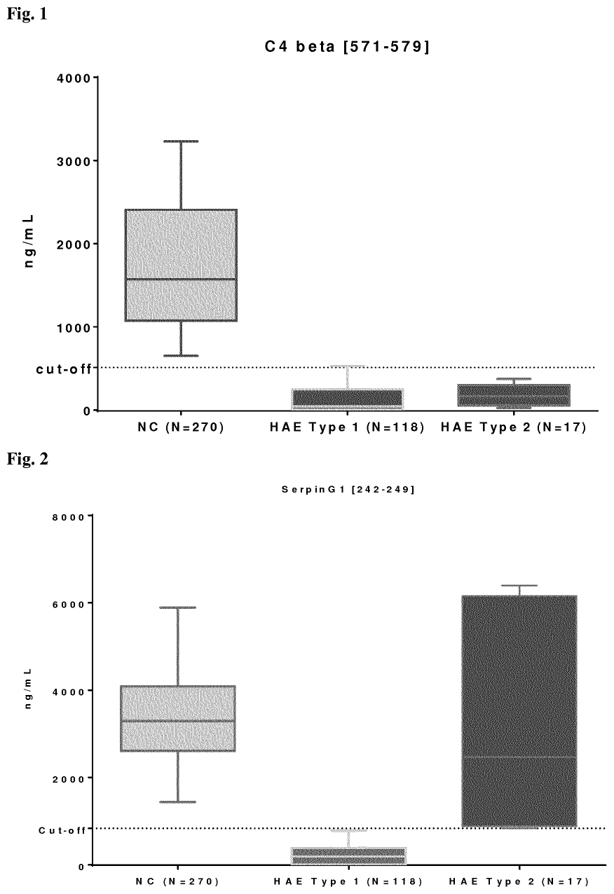

FIGS. 1 to 9 are boxplots indicating levels of the indicated peptide; the y-axis demonstrates the logarithmised levels of said indicated peptide in ng/ml as determined from a dried blood spot on a filter card as described in the Example part; the x-axis depicts groups of subjects which have been grouped as described in the Example part. The boxplot represents the 25th and 75th percentile of each group of subjects by the bottom and top of the box, respectively; the band near the middle of the box represents the 50th percentile (i.e. the median) of each group; the whiskers represent one standard deviation above and below the mean of the data; any data not included between the whiskers is shown as an outlier with a small circle or star. The horizontal line represents the cut-off level of expressed as ng/ml for the indicated peptide.

FIG. 1 is a boxplot of peptide fragment C4Beta_[571-579] of protein C4 beta illustrating a cut-off of 500 ng/ml. Such cut-off allows to distinguish between healthy controls and patients suffering from HAE type 1 and HAE type 2.

FIG. 2 is a boxplot of peptide fragment SerpinG1_[242-249] of protein C1-INH illustrating a cut-off of 835 ng/ml. Such cut-off allows to distinguish between healthy controls and patients suffering from HAE type 1.

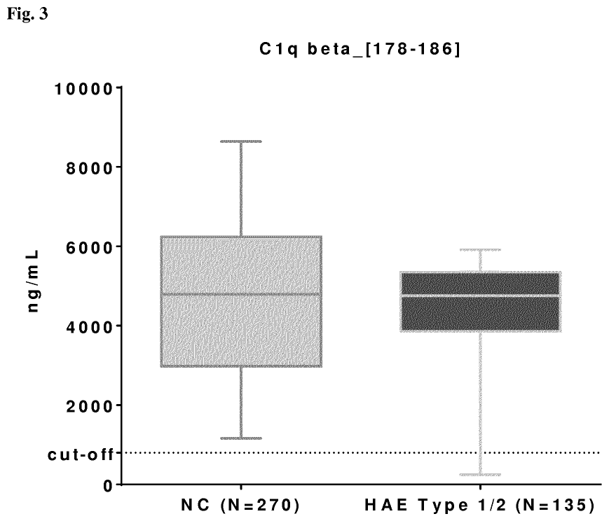

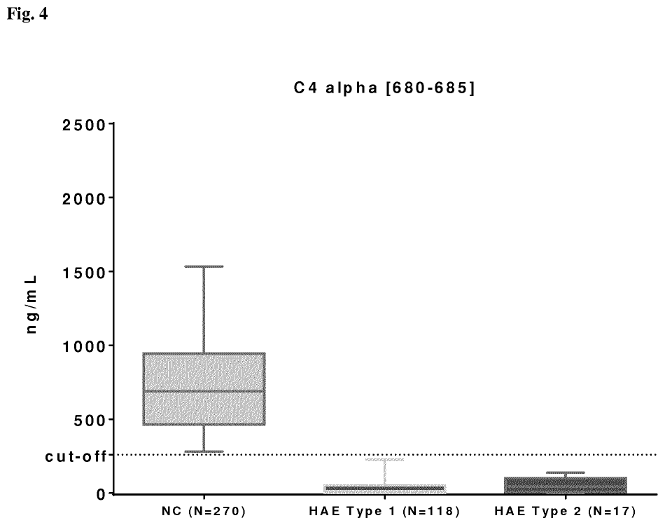

FIG. 3 is a boxplot of peptide fragment C1q Beta_[178-186] of protein C1q beta illustrating a cut-off of 800 ng/ml. This peptide fragment may be used as a control.

FIG. 4 is a boxplot of peptide fragment C4Alpha_[680-685] of protein C4alpha illustrating a cut-off of 260 ng/ml. Such cut-off allows to distinguish between healthy controls and patients suffering from either HAE type 1 or HAE type 2.

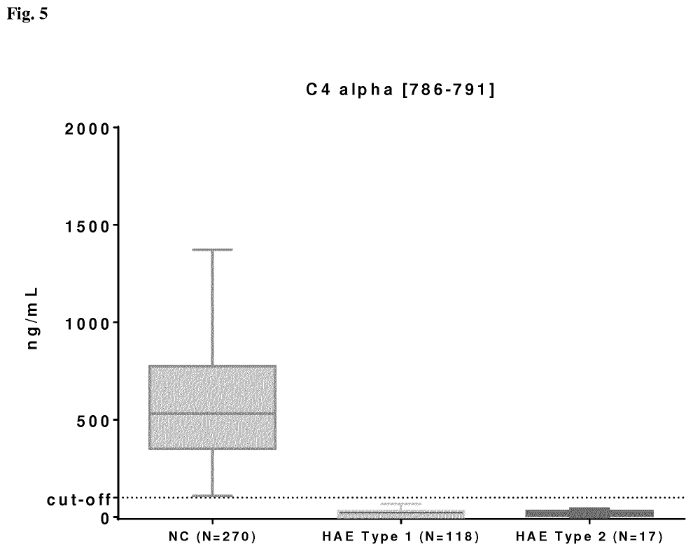

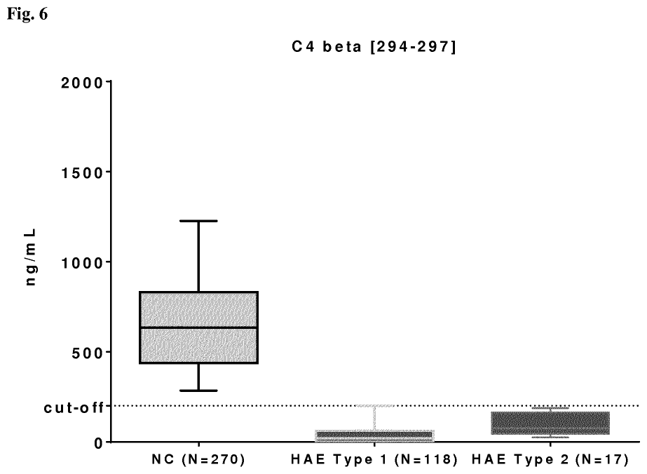

FIG. 5 is a boxplot of peptide fragment C4Alpha_[786-791] of protein C4alpha illustrating a cut-off of 100 ng/ml. Such cut-off allows to distinguish between healthy controls and patients suffering from either HAE type 1 or HAE type 2.

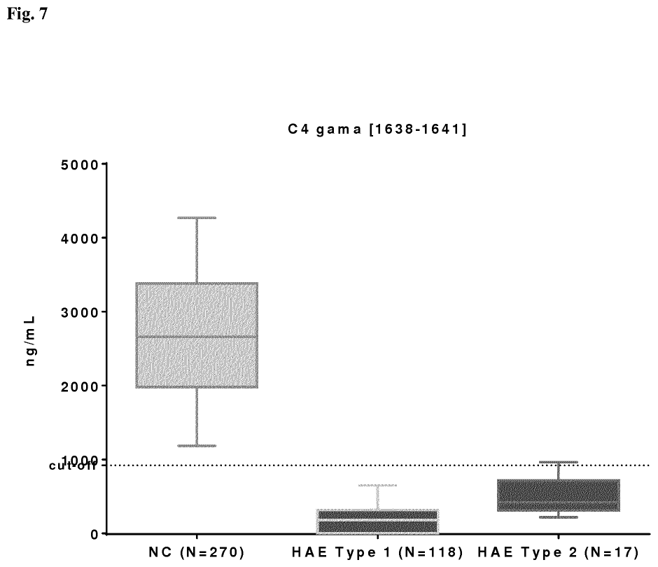

FIG. 6 is a boxplot of peptide fragment C4Beta_[294-297] of protein C4beta illustrating a cut-off of 201 ng/ml. Such cut-off allows to distinguish between healthy controls and patients suffering from either HAE type 1 or HAE type 2.

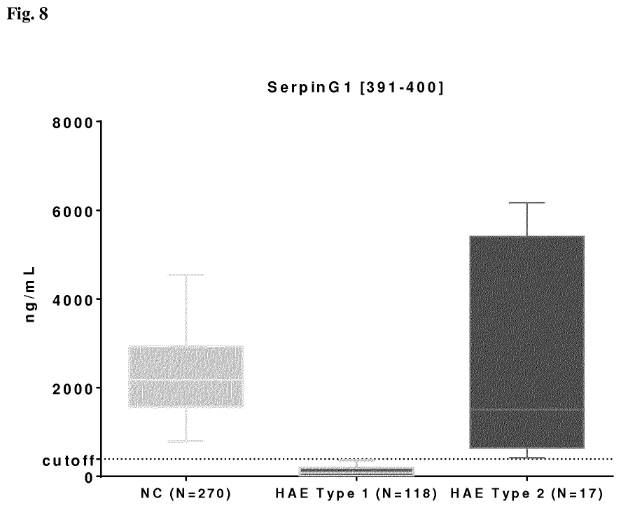

FIG. 7 is a boxplot of peptide fragment C4Gamma_[1638-1641] of protein C4gamma illustrating a cut-off of 920 ng/ml. Such cut-off allows to distinguish between healthy controls and patients suffering from either HAE type 1 or HAE type 2.

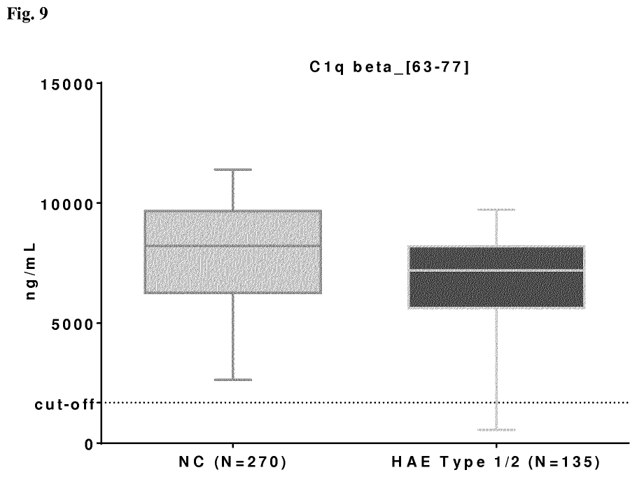

FIG. 8 is a boxplot of peptide fragment SerpinG1_[391-400] of protein C1-INH illustrating a cut-off of 392 ng/ml. Such cut-off allows to distinguish between healthy controls and patients suffering from HAE type 1.

FIG. 9 is a boxplot of peptide fragment C1q-Beta_63-77] of protein C1q beta illustrating a cut-off of 1690 ng/ml. This peptide fragment may be used as a control.

### EXAMPLES

In the Examples described in the following a dried blood spot (abbr. DBS) on a filter card was used as a sample from a subject.

### Example 1: Method for HAE Diagnostic Based on Fragmentation of C3, C1q, C4 and C1-INH into Peptides and Mass Spectrometry Thereof

To quantify the content/levels of C3, C1q, C4 and C1-INH in dried blood spots (DBS) extract a protocol as described was used. After extraction of blood components, the DBS extract was subjected in situ to reaction with dithiothreitol (DDT) to reduce the disulfide bridges in the proteins and to iodacetamide (IAA) to alkylate the free —SH groups. The reaction mixture was then digested in its entirety with trypsin. The tryptic mixture containing peptide fragments of the proteins to be analyzed was injected in LC/IM-high resolution mass spectrometry. For all proteins, peptides without post-transactional modifications could be identified in blood matrix (see Table 1 “C3, C1q, C4 and C1-INH peptides identified in tryptic mixture obtained after total tryptic digestion of DBS extract, selected peptides with +H adducts”). For all peptides fragmentation spectra were obtained and, based on the experimental fragmentation pattern, transitions to be used in multiple reaction monitoring mass spectrometry.

The tryptic peptides could be measured next by LC/MRM-MS. Below are example of tryptic peptides from C3, C1q, C4 and C1-INH detected and quantified using LC/MRM-MS.

Equipment

For detecting the tryptic peptides of the proteins to be quantified in a biological sample of a donor, the following equipment was used:

| Equipment | Model | Provider |
|---|---|---|
| DBS Puncher | 1296-071 Delfia | Perkin Elmer |
| Pipettes | single and multichannel | Eppendorf |
| Vortexer | Mixer UZUSIO VTX-300L | LMS co. LTD |
| Sonicator | SW12H | Sonoswiss |
| Incubator | Titramax 1000 | Heidolph |
| Centrifuge | Benchtop | Eppendorf |
| UPLC | Acuity iclass | Waters |
| IM-qToF | Vion | Waters |
| TQ | TQS-micro | Waters |
| Data mining tool | Progenesis | Nonlinear |

Reagents

For detecting the peptides of the proteins to be quantified in a sample from a subject the following reagents were used. To the extent that values depend on temperature (e.g., the pH value) such values were determined at a temperature of 25° C.

| Name | Supplier | Purity |
|---|---|---|
| 1,4-Dithiothreitol | Roche | >97% |
| Acetonitrile, waterfreei (max. 0.003% H₂O) | VWR | UPLC/ |
| HiPerSolv CHROMANORM ® | | UHPLC |
| | | grade |
| AcroPrep ™ Advance 96 well filter | PALL | |
| platesfor aqueous filtration, 350 μl, 1.0 μm | | |
| glass fibre | | |
| Ammonia solution | Merck | 25% |
| Ammoniumbicarbonate | ACROS | 98% |
| Formic acid, ACS | VWR | >96% |
| Iodoacetamide, IAA | Sigma Aldrich | >99% |
| Kinetex colums EVO C18 | VWR | |
| Leucine-Enkephalin | waters | |
| Methanol HiPerSolv CHROMANORM ® | VWR | LC-MS |
| | | grade |
| Mirco-Platte 96-wells, PP, F-GREINER | VWR | |
| (100 pieces) Natur | | |
| SafeSeal vial 1.5 ml | Sarstedt | |
| Taurocholic acid sodium salt hydrate | Sigma Aldrich | >95% |
| Trypsin 20 μg/vial | Promega | sequencing |
| | | grade |
| Verex ™ Cap (pre-assembled), 8-425, | Phenomenex | |
| Screw top, w/PTFE/Silicone septa, black | | |
| Verex ™ Insert, 5 mm Dia., 175 μL, Clear | Phenomenex | |
| 51, Conical Bottom, w/bottom spring | | |
| Verex ™ Vial, 8 mm Screw Top, 2 mL, | Phenomenex | |
| Clear 33, w/Patch | | |
| Water HiPerSolv ® CHROMANORM ® | VWR | LC-MS |
| | | grade |
| Pipette tips | Sarstedt | |

Preparation of Stock Solution of Internal Standard

Internal Standard (IS 1) stock solution was used as internal standard and was prepared by dissolving 3 mg Leucine-Enkephaline (as provided by Waters, UK) in water to a concentration of 400 μg/mL.

Storing of Samples and Solutions

Control samples and study samples (dried blood spots) were stored at RT. Internal Standard working solutions were stored at room temperature until use.

Sample Preparation for Analysis

1 punch Ø of 3.2 mm was cut from the filter card with dried blood spots and subjected the following protocol:

First, for extraction 100 μL 1 M NH₄HCO₃ were added to the punches, whereby the material was sonicated for 10 min. at 60° C., incubated for 30 min on a shaker (at 700 rpm) at 37° C.

Second, to the solution 125 μl 1 M DTT was added and the reaction mixture was incubated for 3 h at 37° C. on a shaker (700 rpm).

Third, 375 μl 1 M IAA was added and the solution was incubated for 1.5 hours on a shaker (700 rpm) in the dark.

Fourth, 10 μl 0.5 μg/μl trypsin was added and the solution was incubated for 3 to 16 hours on a shaker (700 rpm) in the dark.

The thus obtained solution containing a digest of blood extract was transferred to a PTFE (polytetrafluoroethylene) filter plate (Acroprep™, Pall, Germany) and then to a 96 well plate by centrifugation at 3.500 rpm. Afterwards, 100 μL of internal standard with a known concentration of 20 to 400 ng/mL was added.

Methods

A person skilled in the art will acknowledge that methods for detecting the fragment peptides of the proteins to be analyzed in a sample from a subject using mass spectrometric analysis may also employ other tryptic peptides, specific transitions and specific fragments which allow for specific detection of and/or quantification of HAE relevant peptide fragments and their isoforms in said sample from a subject.

LC/IM-QToF-MS analyses of the peptide fragments of the proteins to be analyzed from DBS extracts were performed using a Waters Acquity iclass UPLC (Waters, UK) coupled with Vion mass spectrometer (Waters, UK) as follows.
1. Chromatographic run was performed on a Kinetex EVO C18 column (Phenomenex, Germany). 10 μL of the extract were injected onto the column and the compounds of the extract were eluted using a linear gradient from 0% A (50 mM formic acid in water) to 100% B (50 mM formic acid in acetonitrile:methanol vol. 1:1).
2. Internal standard was continuously injected at a concentration of 200 ng/mL in water and the signal was used to normalize the sample signal across the batch.

IM-QToF MS analyses were performed in positive ion mode using the following parameters:
Analyzer mode: sensitivity
MS mode: High definition MSE
Capillary voltage: 1.2 kV
Source temperature: 150° C.
Desolvation temperature: 600° C.
Desolvation gas; 1000 L/h
Cone gag: 50 L/h
Low Collision Energy: 6 eV
High Collision Energy Ramp: 20-40 eV
Scan mass: 50-1000 m/z
Scan time: 0.5 s

LC/MRM-MS analyses of the peptide fragments of the proteins to be analyzed for DBS extracts were performed using a Waters Acquity iclass UPLC (Waters, UK) coupled with a TQ-S micro mass spectrometer (Waters, UK).

For the examples the following parameters were used in the quantification of the peptide fragments:
1. Chromatographic run was performed on a Kinetex EVO C18 column (Phenomenex, Germany). The 10 μL extract were injected on the column and the compounds were eluted using a linear gradient from 0% A (50 mM formic acid in water) to 100% B (50 mM formic acid in acetonitrile:methanol vol. 1:1).
2. For the internal standard, MRM transition 556.24→119.97 was monitored. For each peptide, specific transition was used as shown in Example 2.

MRM-MS analyses were performed in positive ion mode using the following parameters:
Capillary voltage: 1.2 kV
Cone voltage: 20 V
Source temperature: 150° C.
Desolvation temperature: 600° C.
Desolvation gas: 1000 L/h
Cone gag: 50 L/h
Collision Energy: 20 V
Collision Cell Entrance: 30 eV
Collision Cell Exit: 30 eV.

### Example 2: Quantifying Peptide Fragments of Proteins C1q, C4, C1-INH and C3 in Dried Blood Spots of Healthy Donors

Using the methods outlined in Example 1, the different peptide fragments of proteins C1 q, C4, C1-INH and C3 were quantified using DBS from a total of 270 healthy subjects.

For Complement C1q the following tryptic peptides could be quantified, whereby the numbers in the brackets represents the position of the first amino acid and the last amino acid of the peptide in amino acid sequence of C1q (with the sequences being indicated with the N-terminus being at the left side and the C-terminus being at the right side). Such peptides are shown in Table 1:

TABLE 1

| Peptide | Sequence (N-terminus → C-terminus) |
|---|---|
| C1q-A_[104-110] | GSPGNIK |
| C1q-A_[111-121] | DQPRPAFSAIR |
| C1q-A_[123-150] | NPPMGGNVVIFDTVITNQEEPYQNHSGR |
| C1q-A_[151-180] | FVCTVPGYYYFTFQVLSQWEICLSIVSSSR |
| C1q-A_[151- | FVCTVPGYYYFTFQVLSQWEICLSIVSSSR |
| 180]_Cys_CAM: 153, 172 | |
| C1q-A_[186-195] | SLGFCDTTNK |
| C1q-A_[186- | SLGFCDTTNK |
| 195]_Cys_CAM: 190 | |
| C1q-A_[196-219] | GLFQVVSGGMVLQLQQGDQVWVEK |
| C1q-A_[224-245] | GHIYQGSEADSVFSGFLIFPSA |
| C1q-A_[23-27] | EDLCR |
| C1q-A_[28-32] | APDGK |
| C1q-A_[34-41] | GEAGRPGR |
| C1q-A_[49-60] | GEQGEPGAPGIR |
| C1q-A_[82-94] | VGYPGPSGPLGAR |
| C1q-B_[118-121] | ATQK |
| C1q-B_[137-141] | DQTIR |
| C1q-B_[160-163] | FTCK |
| C1q-B_[164-177] | VPGLYYFTYHASSR |
| C1q-B_[178-186] | GNLCVNLMR |
| C1q-B_[178- | GNLCVNLMR |
| 186]_Cys_CAM: 181 | |
| C1q-B_[194-215] | VVTFCDYAYNTFQVTTGGMVLK |
| C1q-B_[194- | VVTFCDYAYNTFQVTTGGMVLK |
| 215]_Cys_CAM: 198 | |
| C1q-B_[216-229] | LEQGENVFLQATDK |
| C1q-B_[230-253] | NSLLGMEGANSIFSGFLLFPDMEA |
| Clq-B_[28-59] | QLSCTGPPAIPGIPGIPGTPGPDGQPGTPGIK |
| C1q-B_[28- | QLSCTGPPAIPGIPGIPGTPGPDGQPGTPGIK |
| 59]_Cys_CAM: 31 | |
| C1q-B_[63-77] | GLPGLAGDHGEFGEK |
| C1q-B_[78-88] | GDPGIPGNPGK |
| C1q-B_[93-98] | GPMGPK |
| C1q-B_[99-110] | GGPGAPGAPGPK |
| C1q-C_[118-126] | FQSVFTVTR |
| C1q-C_[127-139] | QTHQPPAPNSLIR |
| C1q-C_[140-157] | FNAVLTNPQGDYDTSTGK |
| C1q-C_[162-184] | VPGLYYFVYHASHTANLCVLLYR |
| C1q-C_[162- | VPGLYYFVYHASHTANLCVLLYR |
| 184]_Cys_CAM: 179 | |
| C1q-C_[189-198] | VVTFCGHTSK |
| C1q-C_[189- | VVTFCGHTSK |
| 198]_Cys_CAM: 193 | |
| C1q-C_[199-210] | TNQVNSGGVLLR |
| C1q-C_[211-245] | LQVGEEVWLAVNDYYDMVGIQGSDSVFSGFLLFPD |
| C1q-C_[29-47] | NTGCYGIPGMPGLPGAPGK |
| C1q-C_[29- | NTGCYGIPGMPGLPGAPGK |
| 47]_Cys_CAM: 32 | |
| C1q-C_[48-57] | DGYDGLPGPK |
| C1q-C_[58-69] | GEPGIPAIPGIR |
| C1q-C_[76-86] | GEPGLPGHPGK |
| C1q-C_[87-113] | NGPMGPPGMPGVPGPMGIPGEPGEEGR |

All of the Complement C1q tryptic peptides can be used to differentiate between healthy subjects and hereditary angioedema patients. Two of said peptides, namely C1q-B_[178-186] with MRM transition 510.26→254.58 and C1q-B_[63-77] with MRM transition 495.25→774.5 were used as representative examples (see Example 3).

For complement C3 the following tryptic peptides could be quantified, whereby the numbers in the brackets represents the position of the first amino acid and the last amino acid of the peptide in amino acid sequence of C3 (with the sequences being indicated with the N-terminus being at the left side and the C-terminus being at the right side). Such peptides are shown in Table 2:

TABLE 2

| Peptide | Sequence (N-terminus → C-terminus) |
|---|---|
| C3Beta_[105-119] | FVTVQATFGTQVVEK |
| C3Beta_[120-136] | VVLVSLQSGYLFIQTDK |
| C3Beta_[137-148] | TIYTPGSTVLYR |
| C3Beta_[149-155] | IFTVNHK |
| C3Beta_[156-161] | LLPVGR |
| C3Beta_[162-176] | TVMVNIENPEGIPVK |
| C3Beta_[177-205] | QDSLSSQNQLGVLPLSWDIPELVNMGQWK |
| C3Beta_[208-225] | AYYENSPQQVFSTEFEVK |
| C3Beta_[226-241] | EYVLPSFEVIVEPTEK |
| C3Beta_[23-35] | SPMYSIITPNILR |
| C3Beta_[242-249] | FYYIYNEK |
| C3Beta_[250-258] | GLEVTITAR |
| C3Beta_[259-263] | FLYGK |
| C3Beta_[265-281] | VEGTAFVIFGIQDGEQR |
| C3Beta_[291-304] | IPIEDGSGEVVLSR |
| C3Beta_[306-315] | VLLDGVQNPR |
| C3Beta_[316-322] | AEDLVGK |
| C3Beta_[323-343] | SLYVSATVILHSGSDMVQAER |
| C3Beta_[344-359] | SGIPIVTSPYQIHFTK |
| C3Beta_[363-386] | YFKPGMPFDLMVFVTNPDGSPAYR |
| C3Beta_[36-65] | LESEETMVLEAHDAQGDVPVTVTVHDFPGK |
| C3Beta_[387-408] | VPVAVQGEDTVQSLTQGDGVAK |
| C3Beta_[409-425] | LSINTHPSQKPLSITVR |
| C3Beta_[429-439] | QELSEAEQATR |
| C3Beta_[440-462] | TMQALPYSTVGNSNNYLHLSVLR |
| C3Beta_[463-478] | TELRPGETLNVNFLLR |
| C3Beta_[479-481] | MDR |
| C3Beta_[482-486] | AHEAK |
| C3Beta_[489-497] | YYTYLIMNK |
| C3Beta_[500-502] | LLK |
| C3Beta_[503-505] | AGR |
| C3Beta_[506-508] | QVR |
| C3Beta_[509-530] | EPGQDLVVLPLSITTDFIPSFR |
| C3Beta_[531-544] | LVAYYTLIGASGQR |
| C3Beta_[545-556] | EVVADSVWVDVK |
| C3Beta_[557-566] | DSCVGSLVVK |
| C3Beta_[557- | DSCVGSLVVK |
| 566]_Cys_CAM: 559 | |
| C3Beta_[574-584] | QPVPGQQMTLK |
| C3Beta_[585-592] | IEGDHGAR |
| C3Beta_[616-622] | IVVDVVEK |
| C3Beta_[623-633] | ADIGCTPGSGK |
| C3Beta_[634-657] | DYAGVFSDAGLTFTSSSGQQTAQR |
| C3Beta_[658-667] | AELQCPQPAA |
| C3Beta_[658- | AELQCPQPAA |
| 667]_Cys_CAM: 662 | |
| C3Beta_[67-73] | LVLSSEK |
| C3Beta_[74-94] | TVLTPATNHMGNVTFTIPANR |
| C3Beta_[95-97] | EFK |
| C3Beta_[98-100] | SEK |
| C3cAlpha1_[749-764] | SNLDEDIIAEENIVSR |
| C3cAlpha1_[765-779] | SEFPESWLWNVEDLK |
| C3cAlpha1_[780-783] | EPPK |
| C3cAlpha1_[784-789] | NGISTK |
| C3cAlpha1_[797-812] | DSITTWEILAVSMSDK |
| C3cAlpha1_[814-834] | GICVADPFEVTVMQDFFIDLR |
| C3cAlpha1_[814- | GICVADPFEVTVMQDFFIDLR |
| 834]_Cys_CAM: 816 | |
| C3cAlpha1_[835-841] | LPYSVVR |
| C3cAlpha1_[842-848] | NEQVEIR |
| C3cAlpha1_[849-855] | AVLYNYR |
| C3cAlpha1_[856-861] | QNQELK |
| C3cAlpha1_[864-879] | VELLHNPAFCSLATTK |
| C3cAlpha1_[864- | VELLHNPAFCSLATTK |
| 879]_Cys_CAM: 873 | |
| C3cAlpha1_[905-913] | TGLQEVEVK |
| C3cAlpha1_[914-926] | AAVYHHFISDGVR |
| C3cAlpha1_[938-940] | MNK |
| C3cAlpha1_[941-945] | TVAVR |
| C3cAlpha1_[946-951] | TLDPER |
| C3cAlpha1_[952-954] | LGR |
| C3cAlpha2_[1321-1325] | SEETK |
| C3cAlpha2_[1326-1337] | ENEGFTVTAEGK |
| C3cAlpha2_[1338-1351] | GQGTLSVVTMYHAK |
| C3cAlpha2_[1354-1360] | DQLTCNK |
| C3cAlpha2_[1354- | DQLTCNK |
| 1360]_Cys_CAM: 1358 | |
| C3cAlpha2_[1361-1364] | FDLK |
| C3cAlpha2_[1365-1375] | VTIKPAPETEK |
| C3cAlpha2_[1376-1381] | RPQDAK |
| C3cAlpha2_[1382-1391] | NTMILEICTR |
| C3cAlpha2_[1382- | NTMILEICTR |
| 1391]_Cys_CAM: 1389 | |
| C3cAlpha2_[1394-1419] | GDQDATMSILDISMMTGFAPDTDDLK |
| C3cAlpha2_[1420-1427] | QLANGVDR |
| C3cAlpha2_[1428-1431] | YISK |
| C3cAlpha2_[1432-1436] | YELDK |
| C3cAlpha2_[1437-1441] | AFSDR |
| C3cAlpha2_[1442-1450] | NTLIIYLDK |
| C3cAlpha2_[1451-1462] | VSHSEDDCLAFK |
| C3cAlpha2_[1451- | VSHSEDDCLAFK |
| 1462]_Cys_CAM: 1458 | |
| C3cAlpha2_[1463-1478] | VHQYFNVELIQPGAVK |
| C3cAlpha2_[1479-1491] | VYAYYNLEESCTR |
| C3cAlpha2_[1479- | VYAYYNLEESCTR |
| 1491]_Cys_CAM: 1489 | |
| C3cAlpha2_[1492-1497] | FYHPEK |
| C3cAlpha2_[1502-1504] | LNK |
| C3cAlpha2_[1505-1507] | LCR |
| C3cAlpha2_[1505- | LCR |
| 1507]_Cys_CAM: 1506 | |
| C3cAlpha2_[1527-1532] | VTLEER |
| C3cAlpha2_[1533-1535] | LDK |
| C3cAlpha2_[1536-1546] | ACEPGVDYVYK |
| C3cAlpha2_[1536- | ACEPGVDYVYK |
| 1546]_Cys_CAM: 1537 | |
| C3cAlpha2_[1552-1570] | VQLSNDFDEYIMAIEQTIK |
| C3cAlpha2_[1571-1582] | SGSDEVQVGQQR |
| C3cAlpha2_[1583-1589] | TFISPIK |
| C3cAlpha2_[1592-1595] | EALK |
| C3cAlpha2_[1596-1599] | LEEK |
| C3cAlpha2_[1601-1624] | HYLMWGLSSDFWGEKPNLSYIIGK |
| C3cAlpha2_[1625-1644] | DTWVEHWPEEDECQDEENQK |
| C3cAlpha2_[1625- | DTWVEHWPEEDECQDEENQK |
| 1644]_Cys_CAM: 1637 | |

All of the Complement C3 tryptic peptides can be used in the assay. Two of said peptides, namely C3Beta_[489-497] with MRM transition 604.8→327.22 and C3cAlpha1_[814-834]_Cys_CAM: 816 with MRM transition 824.74→798.44 were used as illustrative examples (see Example 3).

For complement C4 the following tryptic peptides could be quantified, whereby the numbers in the brackets represents the position of the first amino acid and the last amino acid of the peptide in amino acid sequence of C4 (with the sequences being indicated with the N-terminus being at the left side and the C-terminus being at the right side). Such peptides are shown in Table 3.

TABLE 3

| Peptide | Sequence (N-terminus → C-terminus) |
|---|---|
| C4Alpha_[1006-1008] | LPR |
| C4Alpha_[1009-1026] | GCGEQTMIYLAPTLAASR |
| C4Alpha_[1009- | GCGEQTMIYLAPTLAASR |
| 1026]_Cys_CAM: 1010 | |
| C4Alpha_[1027-1030] | YLDK |
| C4Alpha_[1031-1042] | TEQWSTLPPETK |
| C4Alpha_[1043-1051] | DHAVDLIQK |
| C4Alpha_[1052-1055] | GYMR |
| C4Alpha_[1062-1072] | ADGSYAAWLSR |
| C4Alpha_[1073-1084] | GSSTWLTAFVLK |
| C4Alpha_[1085-1099] | VLSLAQEQVGGSPEK |
| C4Alpha_[1100-1126] | LQETSNWLLSQQQADGSFQDLSPVIHR |
| C4Alpha_[1168-1174] | VEASISK |
| C4Alpha_[1175-1182] | ASSFLGEK |
| C4Alpha_[1183-1204] | ASAGLLGAHAAAITAYALTLTK |
| C4Alpha_[1211-1248] | GVAHNNLMAMAQETGDNLYWGSVTGSQSNA |
| | VSPTPAPR |
| C4Alpha_[1249-1278] | NPSDPMPQAPALWIETTAYALLHLLLHEGK |
| C4Alpha_[1279-1291] | AEMADQAAAWLTR |
| C4Alpha_[1292-1300] | QGSFQGGFR |
| C4Alpha_[1301-1325] | STQDTVIALDALSAYWIASHTTEER |
| C4Alpha_[1326-1336] | GLNVTLSSTGR |
| C4Alpha_[1337-1340] | NGFK |
| C4Alpha_[1341-1349] | SHALQLNNR |
| C4Alpha_[1350-1352] | QIR |
| C4Alpha_[1353-1365] | GLEEELQFSLGSK |
| C4Alpha_[1370-1375] | VGGNSK |
| C4Alpha_[1383-1390] | TYNVLDMK |
| C4Alpha_[1391-1404] | NTTCQDLQIEVTVK |
| C4Alpha_[1391- | NTTCQDLQIEVTVK |
| 1404]_Cys_CAM: 1394 | |
| C4Alpha_[1405-1428] | GHVEYTMEANEDYEDYEYDELPAK |
| C4Alpha_[1429-1446] | DDPDAPLQPVTPLQLFEG |
| C4Alpha_[680-685] | NVNFQK |
| C4Alpha_[686-690] | AINEK |
| C4Alpha_[691-700] | LGQYASPTAK |
| C4Alpha_[702- | CCQDGVTR |
| 709]_Cys_CAM: 702, 703 | |
| C4Alpha_[710-714] | LPMMR |
| C4Alpha_[715- | SCEQR |
| 719]_Cys_CAM: 716 | |
| C4Alpha_[723-729] | VQQPDCR |
| C4Alpha_[723- | VQQPDCR |
| 729]_Cys_CAM: 728 | |
| C4Alpha_[730- | EPFLSCCQFAESLR |
| 743]_Cys_CAM: 735, 736 | |
| C4Alpha_[750-756] | GQAGLQR |
| C4Alpha_[757-775] | ALEILQEEDLIDEDDIPVR |
| C4Alpha_[776-785] | SFFPENWLWR |
| C4Alpha_[786-791] | VETVDR |
| C4Alpha_[792-815] | FQILTLWLPDSLTTWEIHGLSLSK |
| C4Alpha_[818-828] | GLCVATPVQLR |
| C4Alpha_[818- | GLCVATPVQLR |
| 828]_Cys_CAM: 820 | |
| C4Alpha_[832-838] | EFHLHLR |
| C4Alpha_[846-861] | FEQLELRPVLYNYLDK |
| C4Alpha_[862-912] | NLTVSVHVSPVEGLCLAGGGGLAQQVLVPAGS |
| | ARPVAFSVVPTAATAVSLK |
| C4Alpha_[862- | NLTVSVHVSPVEGLCLAGGGGLAQQVLVPAGS |
| 912]_Cys_CAM: 876 | ARPVAFSVVPTAATAVSLK |
| C4Alpha_[913-916] | VVAR |
| C4Alpha_[917-929] | GSFEFPVGDAVSK |
| C4Alpha_[936-941] | EGAIHR |
| C4Alpha_[942-954] | EELVYELNPLDHR |
| C4Alpha_[957-979] | TLEIPGNSDPNMIPDGDFNSYVR |
| C4Alpha_[980-1005] | VTASDPLDTLGSEGALSPGGVASLLR |
| C4Beta_[105-118] | GPEVQLVAHSPWLK |
| C4Beta_[119-123] | DSLSR |
| C4Beta_[124-137] | TTNIQGINLLFSSR |
| C4Beta_[139-155] | GHLFLQTDQPIYNPGQR |
| C4Beta_[158-159] | YR |
| C4Beta_[160-166] | VFALDQK |
| C4Beta_[167-185] | MRPSTDTITVMVENSHGLR |
| C4Beta_[190-214] | EVYMPSSIFQDDFVIPDISEPGTWK |
| C4Beta_[219-234] | FSDGLESNSSTQFEVK |
| C4Beta_[23-48] | LLLFSPSVVHLGVPLSVGVQLQDVPR |
| C4Beta_[236-244] | YVLPNFEVK |
| C4Beta_[245-269] | ITPGKPYILTVPGHLDEMQLDIQAR |
| C4Beta_[270-283] | YIYGKPVQGVAYVR |
| C4Beta_[284-292] | FGLLDEDGK |
| C4Beta_[294-297] | TFFR |
| C4Beta_[298-304] | GLESQTK |
| C4Beta_[305-316] | LVNGQSHISLSK |
| C4Beta_[326-337] | LNMGITDLQGLR |
| C4Beta_[338-373] | LYVAAAIIESPGGEMEEAELTSWYFVSSPFSLDL |
| | SK |
| C4Beta_[392-404] | EMSGSPASGIPVK |
| C4Beta_[405-459] | VSATVSSPGSVPEVQDIQQNTDGSGQVSIPIIIPQ |
| | TISELQLSVSAGSPHPAIAR |
| C4Beta_[460-484] | LTVAAPPSGGPGFLSIERPDSRPPR |
| C4Beta_[485-494] | VGDTLNLNLR |
| C4Beta_[49-53] | GQVVK |
| C4Beta_[495-512] | AVGSGATFSHYYYMILSR |
| C4Beta_[513-520] | GQIVFMNR |
| C4Beta_[521-523] | EPK |
| C4Beta_[525-559] | TLTSVSVFVDHHLAPSFYFVAFYYHGDHPVANS |
| | LR |
| C4Beta_[560-570] | VDVQAGACEGK |
| C4Beta_[560- | VDVQAGACEGK |
| 570]_Cys_CAM: 567 | |
| C4Beta_[571-579] | LELSVDGAK |
| C4Beta_[580-582] | QYR |
| C4Beta_[583-588] | NGESVK |
| C4Beta_[589-614] | LHLETDSLALVALGALDTALYAAGSK |
| C4Beta_[60-63] | NPSR |
| C4Beta_[615-623] | SHKPLNMGK |
| C4Beta_[624-664] | VFEAMNSYDLGCGPGGGDSALQVFQAAGLAFS |
| | DGDQWTLSR |
| C4Beta_[624- | VFEAMNSYDLGCGPGGGDSALQVFQAAGLAFS |
| 664]_Cys_CAM: 635 | DGDQWTLSR |
| C4Beta_[64-71] | NNVPCSPK |
| C4Beta_[64-71]_Cys_CAM: 68 | NNVPCSPK |
| C4Beta_[667-671] | LSCPK |
| C4Beta_[667- | LSCPK |
| 671]_Cys_CAM: 669 | |
| C4Beta_[72-80] | VDFTLSSER |
| C4Beta_[81-92] | DFALLSLQVPLK |
| C4Beta_[93-95] | DAK |
| C4Beta_[96-104] | SCGLHQLLR |
| C4Beta_[96-104]_Cys_CAM: 97 | SCGLHQLLR |
| C4Gamma_[1458-1465] | VVEEQESR |
| C4Gamma_[1466-1474] | VHYTVCIVVR |
| C4Gamma_[1466- | VHYTVCIVVR |
| 1474]_Cys_CAM: 1471 | |
| C4Gamma_[1475-1477] | NGK |
| C4Gamma_[1478-1498] | VGLSGMAIADVTLLSGFHALR |
| C4Gamma_[1499-1503] | ADLEK |
| C4Gamma_[1504-1510] | LTSLSDR |
| C4Gamma_[1511-1533] | YVSHFETEGPHVLLYFDSVPTSR |
| C4Gamma_[1534-1564] | ECVGFEAVQEVPVGLVQPASATLYDYYNPER |
| C4Gamma_[1534- | |
| 1564]_Cys_CAM: 1535 | ECVGFEAVQEVPVGLVQPASATLYDYYNPER |
| C4Gamma_[1566-1575] | CSVFYGAPSK |
| C4Gamma_[1566- | CSVFYGAPSK |
| 1575]_Cys_CAM: 1566 | |
| C4Gamma_[1578-1594 | LLATLCSAEVCQCAEGK |
| C4Gamma_[1578- | LLATLCSAEVCQCAEGK |
| 1594]_Cys_CAM: 1583, 1588, | |
| 1590 | |
| C4Gamma_[1595-1597] | CPR |
| C4Gamma_[1595- | |
| 1597]_Cys_CAM: 1595 | CPR |
| C4Gamma_[1601-1604] | ALER |
| C4Gamma_[1616-1622] | FACYYPR |
| C4Gamma_[1616- | FACYYPR |
| 1622]_Cys_CAM: 1618 | |
| C4Gamma_[1623-1630] | VEYGFQVK |
| C4Gamma_[1631-1633] | VLR |
| C4Gamma_[1638-1641] | AAFR |
| C4Gamma_[1642-1646] | LFETK |
| C4Gamma_[1656-1658] | DVK |
| C4Gamma_[1659-1665] | AAANQMR |
| C4Gamma_[1671-1674] | ASCR |
| C4Gamma_[1677-1681] | LEPGK |
| C4Gamma_[1682-1716] | EYLIMGLDGATYDLEGHPQYLLDSNSWIEEMPS |
| | ER |
| C4Gamma_[1720-1722] | STR |
| C4Gamma_[1725-1744] | AACAQLNDFLQEYGTQGCQV |
| C4Gamma_[1725- | AACAQLNDFLQEYGTQGCQV |
| 1744]_Cys_CAM: 1727, 1742 | |

All of the Complement C4 tryptic peptides can be used to differentiate between healthy subjects and hereditary angioedema patients. for of the peptides, namely C4Alpha_[680-685] with MRM transition 375.2→536.28, C4Alpha_[786-791] with MRM transition 359.69→490.26, C4Beta_[294-297] with MRM transition 285.66→322.19, C4Beta_[571-579] with MRM transition 466.26→243.13, and C4 gamma_[1638-1641] with MRM transition 232.64→322.19] were used as representative examples (see Example 3).

For complement C1-INH (also referred to as SerpinG1), the following tryptic peptides could be quantified, whereby the numbers in the brackets represents the position of the first amino acid and the last amino acid of the peptide in amino acid sequence of C1-INH (with the sequences being indicated with the N-terminus being at the left side and the C-terminus being at the right side). Such peptides are shown in Table 4.

TABLE 4

| Peptide | Sequence (N-terminus → C-terminus) |
|---|---|
| SerpinG1_[202-211 | DFTCVHQALK |
| SerpinG1_[202- | DFTCVHQALK |
| 211]_Cys_CAM: 205 | |
| SerpinG1_[212-216] | GFTTK |
| SerpinG1_[217-233] | GVTSVSQIFHSPDLAIR |
| SerpinG1_[23-40] | NPNATSSSSQDPESLQDR |
| SerpinG1_[234-241] | DTFVNASR |
| SerpinG1_[242-249] | TLYSSSPR |
| SerpinG1_[250-268] | VLSNNSDANLELINTWVAK |
| SerpinG1_[269-273] | NTNNK |
| SerpinG1_[274-276] | ISR |
| SerpinG1_[277-286] | LLDSLPSDTR |
| SerpinG1_[301-306] | TTFDPK |
| SerpinG1_[310-316] | MEPFHFK |
| SerpinG1_[322-328] | VPMMNSK |
| SerpinG1_[330-341] | YPVAHFIDQTLK |
| SerpinG1_[344-364] | VGQLQLSHNLSLVILVPQNLK |
| SerpinG1_[367-380] | LEDMEQALSPSVFK |
| SerpinG1_[381-385] | AIIVIEK |
| SerpinG1_[386-390] | LEMSK |
| SerpinG1_[391-400] | FQPTLLTLPR |
| SerpinG1_[403-415] | VTTSQDMLSIMEK |
| SerpinG1_[41-44] | GEGK |
| SerpinG1_[416-466] | LEFFDFSYDLNLCGLTEDPDLQVSAMQHQTVLELTET |
| | GVEAAAASAISVAR |
| SerpinG1_[467-487] | TLLVFEVQQPFLFVLWDQQHK |
| SerpinG1_[488-494] | FPVFMGR |
| SerpinG1_[495-499] | VYDPR |
| SerpinG1_[53-77] | MLFVEPILEVSSLPTTNSTTNSATK |

All the complement C1-INH tryptic peptides can be used to differentiate between healthy subjects and hereditary angioedema patients. Two peptides, namely SerpinG1_[242-249] with MRM transition 455.74→696.33 and SerpinG1 [391-400] with MRM transition 593.35→455.79], were used as representative example (see Example 3).

### Example 3: Quantifying Tryptic Peptide Fragments of the Proteins C3, C1q, C4 and C1-INH in DBS Extract from Healthy Subjects and Hereditary Angioedema Patients with Known Pathogenic Variants in Serping1 Gene

HAE Patients

All patients with hereditary angioedema disease type 1/2 or of whom it was strongly assumed that they were suffering from hereditary angioedema disease type 1/2 sent to the participating centers were included into the study. SerpinG1 mutations were confirmed in all the patients taken in consideration for this study using techniques such as next generation sequencing, Sanger sequencing and/or multiplexed ligation dependent probe amplification.

Protein C4

Using the methods outlined in Example 1, the content of peptide fragments C4Alpha_[680-685], C4Alpha_[786-791], C4Beta_[294-297], C4Beta_[571-579] and C4 gamma_[1638-1641] of protein C4 was quantified in DBS from a total of 270 healthy subjects. Similarly, the content of peptide fragments C4Alpha_[680-685], C4Alpha_[786-791], C4Beta_[294-297], C4Beta_[571-579] and C4 gamma_[1638-1641] of the protein C4 was quantified in DBS from a total of 135 previously genetically diagnosed hereditary angioedema patients.

For this assay, pure synthetic peptides were obtained and used to obtain a standard curve used to quantify the peptides originating from blood samples. The linearity of the standard curve is reflected in R² values in the following Table 5.

TABLE 5

| | | | Parent | | | |
|---|---|---|---|---|---|---|
| | | | ion | | | |
| | | Molecular | charge | | Retention | R² |
| Protein | Sequence | weight | state | Transition | time | Linearity |
| C4Beta | LELSVDGAK | 930.50 | 2+ | 466.26/243.13 | 3.5 | 0.998109 |
| [571-579] | | | | | | |
| C4Alpha | NVNFQK | 748.39 | 2+ | 375.2/536.28 | 2.4 | 0.999882 |
| [680-685] | | | | | | |
| C4Alpha | VETVDR | 717.37 | 2+ | 359.69/490.26 | 2 | 0.999694 |
| [786-791] | | | | | | |
| C4Beta | TFFR | 569.30 | 2+ | 285.66/322.19 | 3.1 | 0.987227 |
| [294-297] | | | | | | |
| C4Gamma | AAFR | 463.25 | 2+ | 232.64/322.19 | 2 | 0.999615 |
| [1638-1641] | | | | | | |

As shown in Table 6 below, peptides C4Alpha[680-685], C4Alpha_[786-791], C4Beta_[294-297], C4Beta_[571-579] and C4Gamma_[1638-1641] of protein C4 were reduced in a statistically significant manner in hereditary angioedema patients compared to healthy subjects (p<0,0001). The values for the various peptides are in ng/ml.

TABLE 6

| | |
|---|---|
| HAE | HAE |

| | | Controls | Type 1 | Type 2 |
|---|---|---|---|---|
| Peptide | Number of values (N) | 270 | 118 | 17 |
| C4Beta_[571- | Minimum | 500 | 6.25 | 25 |
| 579] | 25% Percentile | 1075 | 6.25 | 56.25 |
| | Median | 1572 | 37.5 | 165.6 |
| | 75% Percentile | 2408 | 228.1 | 298.4 |
| | Maximum | 3400 | 475 | 375 |
| | Mean | 1760 | 114.3 | 173 |
| | Std. Deviation | 770.2 | 131.3 | 122.5 |
| | Std. Error of Mean | 47.22 | 12.3 | 30.62 |
| | Lower 95% CI of mean | 1667 | 89.94 | 107.8 |
| | Upper 95% CI of mean | 1853 | 138.7 | 238.3 |
| C4Alpha_[680- | Minimum | 268.8 | 0 | 0 |
| 685] | 25% Percentile | 462.5 | 0 | 6.25 |
| | Median | 687.5 | 12.5 | 43.75 |
| | 75% Percentile | 943.8 | 50 | 100 |
| | Maximum | 2131 | 237.5 | 137.5 |
| | Mean | 723.5 | 36.74 | 52.21 |
| | Std. Deviation | 327.7 | 50.34 | 47.18 |
| | Std. Error of Mean | 19.98 | 4.694 | 11.44 |
| | Lower 95% CI of mean | 684.1 | 27.44 | 27.95 |
| | Upper 95% CI of mean | 762.8 | 46.04 | 76.47 |
| C4Alpha_[786- | Minimum | 100 | 0 | 0 |
| 791] | 25% Percentile | 350 | 0 | 7.813 |
| | Median | 531.3 | 12.5 | 18.75 |
| | 75% Percentile | 775 | 31.25 | 31.25 |
| | Maximum | 1444 | 75 | 43.75 |
| | Mean | 590.9 | 16.39 | 19.14 |
| | Std. Deviation | 329.7 | 19.96 | 13 |
| | Std. Error of Mean | 20.49 | 1.87 | 3.251 |
| | Lower 95% CI of mean | 550.5 | 12.69 | 12.21 |
| | Upper 95% CI of mean | 631.2 | 20.1 | 26.07 |
| C4Beta_[294- | Minimum | 218.8 | 0 | 25 |
| 297] | 25% Percentile | 437.5 | 0 | 43.75 |
| | Median | 634.4 | 18.75 | 75 |
| | 75% Percentile | 831.3 | 62.5 | 162.5 |
| | Maximum | 1488 | 200 | 187.5 |
| | Mean | 657.5 | 35.84 | 95.31 |
| | Std. Deviation | 259 | 49.89 | 58.96 |
| | Std. Error of Mean | 15.76 | 4.613 | 14.74 |
| | Lower 95% CI of mean | 626.4 | 26.71 | 63.89 |
| | Upper 95% CI of mean | 688.5 | 44.98 | 126.7 |
| C4Gamma_ | Minimum | 975 | 0 | 218.8 |
| [1638-1641] | 25% Percentile | 1980 | 0 | 309.4 |
| | Median | 2659 | 181.3 | 418.8 |
| | 75% Percentile | 3381 | 318.8 | 715.6 |
| | Maximum | 5550 | 912.5 | 962.5 |
| | Mean | 2704 | 187.7 | 500.8 |
| | Std. Deviation | 969.8 | 214.9 | 233 |
| | Std. Error of Mean | 59.02 | 19.7 | 58.25 |
| | Lower 95% CI of mean | 2588 | 148.6 | 376.6 |
| | Upper 95% CI of mean | 2820 | 226.7 | 624.9 |

Protein C1-INH

Using the methods outlined in Example 1, the content of peptide fragments SerpinG1_[242-249] and SerpinG1_[391-400] of protein C1-INH were quantified in DBS from a total of 270 healthy subjects. Similarly, the content of peptide fragments SerpinG_[242-249] and SerpinG1_[391-400] of protein C1-INH was quantified in DBS from a total of 135 previously genetically diagnosed hereditary angioedema patients.

For this assay, pure synthetic peptides were obtained and used to obtain a standard curve used to quantify the peptides originating from blood samples. The linearity of the standard curve is reflected in R² values in the following Table 7:

TABLE 7

| | | | Parent | | | |
|---|---|---|---|---|---|---|
| | | | ion | | | |
| | | Molecular | charge | | Retention | R² |
| Protein | Sequence | weight | state | Transition | time | Linearity |
| SerpinG1 | TLYSSSPR | 909.46 | 2+ | 455.74/696.33 | 2.6 | 0.999621 |
| [242-249] | | | | | | |
| SerpinG1 | FQPTLLTLPR | 1184.69 | 2+ | 593.35/455.79 | 4.8 | 0.972887 |
| [391-400] | | | | | | |

As shown in Table 8 below, SerpinG_[242-249] and SerpinG1_[391-400] of protein C1-IHN were reduced in a statistically significant manner in hereditary angioedema patients type 1 in comparison to healthy subjects (p<0,0001). The values for the various peptides are in ng/ml.

TABLE 8

| | | | HAE | HAE |
|---|---|---|---|---|
| | Number of | Controls | Type 1 | Type 2 |
| Peptide | values (N) | 270 | 118 | 17 |
| SerpinG1_[242- | Minimum | 1113 | 0 | 837.5 |
| 249] | 25% Percentile | 2606 | 0 | 884.4 |
| | Median | 3291 | 190.6 | 2463 |
| | 75% Percentile | 4081 | 381.3 | 6156 |
| | Maximum | 6475 | 831.3 | 6394 |
| | Mean | 3412 | 223.1 | 2956 |
| | Std. Deviation | 1174 | 228.9 | 2319 |
| | Std. Error of Mean | 71.43 | 21.07 | 562.4 |
| | Lower 95% CI of mean | 3272 | 181.4 | 1764 |
| | Upper 95% CI of mean | 3553 | 264.8 | 4148 |
| SerpinG1_[391- | Minimum | 675 | 0 | 418.8 |
| 400] | 25% Percentile | 1566 | 0 | 640.6 |
| | Median | 2169 | 81.25 | 1500 |
| | 75% Percentile | 2930 | 189.1 | 5406 |
| | Maximum | 5069 | 393.8 | 6169 |
| | Mean | 2322 | 107.4 | 2384 |
| | Std. Deviation | 985.7 | 113.8 | 2284 |
| | Std. Error of Mean | 59.99 | 10.47 | 553.9 |
| | Lower 95% CI of mean | 2204 | 86.62 | 1210 |
| | Upper 95% CI of mean | 2440 | 128.1 | 3558 |

For all samples included in the study, the HAE type 1 patients could be distinguished from healthy controls (non-HAE).

Protein C1q

Using the methods outlined in Example 1, the content of peptide fragments C1q-B_[178-186] and C1q-B_[63-77] of protein C1q were quantified in DBS from a total of 270 healthy subjects.

Similarly, the content of peptide fragments C1q-B_[178-186] and C1q-B_[63-77] of the protein C1q were determined in DBS from a total of 135 previously genetically diagnosed hereditary angioedema patients.

For this assay, pure synthetic peptides were obtained and used to obtain a standard curve used to quantify the peptides originating from blood samples. The linearity of the standard curve is reflected in R² values in the following Table 9.

TABLE 9

| | | | Parent | | | |
|---|---|---|---|---|---|---|
| | | | ion | | | |
| | | Molecular | charge | | Retention | R² |
| Protein | Sequence | weight | state | Transition | time | Linearity |
| C1qBeta | GNLCVNLMR | 1018.51 | 2+ | 510.26/254.58 | 3.9 | 0.983744 |
| [178-186] | | | | | | |
| C1q-B | GLPGLAGD | 1482.7104 | 3+ | 495.25/774.5 | 4.1 | 0.973842 |
| [63-77] | HGEFGEK | | | | | |

As shown in Table 10 below, peptide fragments C1q-B_[178-186] and C1q-B_[63-77] of protein C1q were not significantly reduced in a statistically significant manner in hereditary angioedema patients type 1 compared to healthy subjects. The values for the various peptides are in ng/ml.

TABLE 10

| | | | HAE | HAE |
|---|---|---|---|---|
| | | Controls | Type 1 | Type 2 |
| Peptide | Number of values (N) | 270 | 118 | 17 |

| | | | |
|---|---|---|---|
| C1q-B_[178-186] | Minimum | 1000 | 247 |
| | 25% Percentile | 2982 | 3857 |
| | Median | 4790 | 4750 |
| | 75% Percentile | 6240 | 5355 |
| | Maximum | 9365 | 5916 |
| | Mean | 4698 | 4250 |
| | Std. Deviation | 2062 | 1647 |
| | Std. Error of Mean | 291.6 | 425.3 |
| | Lower 95% CI of mean | 4112 | 3338 |
| | Upper 95% CI of mean | 5284 | 5162 |
| C1q-B_[63-77] | Minimum | 1783 | 552.6 |
| | 25% Percentile | 6262 | 5635 |
| | Median | 8212 | 7196 |
| | 75% Percentile | 9666 | 8200 |
| | Maximum | 11821 | 9729 |
| | Mean | 7651 | 6684 |
| | Std. Deviation | 2652 | 2183 |
| | Std. Error of Mean | 364.2 | 563.7 |
| | Lower 95% CI of mean | 6920 | 5475 |
| | Upper 95% CI of mean | 8382 | 7893 |

Protein C3

Using the methods outlined in Example 1, the content of peptide fragment C3Beta_[250-258] of protein C3 was quantified in DBS from a total of 270 healthy subjects. Similarly, the content of peptide fragment C3Beta_[250-258] of protein C3 was quantified in DBS from a total of 135 previously genetically diagnosed hereditary angioedema patients.

For this assay, pure synthetic peptides were obtained and used to obtain a standard curve used to quantify the peptides originating from blood samples. The linearity of the standard curve is reflected in R² values in the following Table 11.

TABLE 11

| | | | Parent | | | |
|---|---|---|---|---|---|---|
| | | | ion | | | |
| | | Molecular | charge | | Retention | R² |
| Protein | Sequence | weight | state | Transition | time | Linearity |
| C3Beta | GLEVTITAR | 958.54 | 2+ | 480.28/660.4 | 3.7 | 0.943337 |
| [250-258] | | | | | | |

As shown in Table 12 below, C3Beta_[250-258] of the protein C1q is not reduced in a statistically significant matter in hereditary angioedema patients type 1 compared to healthy subjects. The values for the various peptides are in ng/ml.

TABLE 12

| | | | HAE | HAE |
|---|---|---|---|---|
| | | Controls | Type 1 | Type 2 |
| Peptide | Number of values (N) | 270 | 118 | 17 |

| | | | |
|---|---|---|---|
| C3Beta_[250-258] | Minimum | 1 | 0.0 |
| | 25% Percentile | 3.785 | 0.0300 |
| | Median | 6.73 | 2.840 |
| | 75% Percentile | 10.36 | 3.960 |
| | Maximum | 15.78 | 8.710 |
| | Mean | 7.07 | 2.339 |
| | Std. Deviation | 3.923 | 2.347 |
| | Std. Error of Mean | 0.2466 | 0.2020 |
| | Lower 95% CI of mean | 6.584 | 1.940 |
| | Upper 95% CI of mean | 7.555 | 2.739 |

### Example 4: Determination of Biomarker Cut-Off Levels in DBS Extract from Healthy Subjects and Hereditary Angioedema Patients with Known Pathogenic Variants in Serping1 Gene

Based on the data and results of Example 3 and using synthetic peptides as calibration standards for SerpinG_[242-249], C4Alpha_[680-6851, C4Alpha_[786-791], C4Beta_[294-297], C4Beta_[571-579], C1q-Beta_[178-186], C4Gamma_[1638-1641], SerpinG1_[391-400] and C1q-Beta_63-77] a cut-off level was determined empirically for each peptide.

| Peptide | Cut-off |
|---|---|
| C4Beta_[571 -579] | 500 ng/mL |
| SerpinG1_[242-249] | 835 ng/mL |
| C1q-Beta_[178-186] | 800 ng/mL |
| C4Alpha_[680-685] | 260 ng/mL |
| C4Alpha_[786-791] | 100 ng/mL |
| C4Beta_[294-297] | 201 ng/mL |
| C4Gamma_[1638-1641] | 920 ng/mL |
| SerpinG1_[391-400] | 392 ng/mL |
| C1q-Beta_[63-77] | 1690 ng/mL |

The results are shown in FIGS. 1 to 9.

For all samples included in the study, the HAE patients could be distinguished from the healthy controls (non-HAE, NC).

The features of the present invention disclosed in the specification, the claims, the sequence listing and/or the drawings may both separately and in any combination thereof be material for realizing the invention in various forms thereof.

1. A method for differential diagnosis of hereditary angioedema, wherein the method comprises determining the level of C4 protein, C1-INH protein and C1q protein in a sample from a subject, wherein the sample is a dried blood spot sample and wherein the level is determined by mass spectrometry.

2. The method of claim 1, wherein determining the level of C4 protein comprises detecting and quantifying the level of a C4 fragment peptide, wherein determining the level of C1-INH protein comprises detecting and quantifying the level of a C1-INH fragment peptide, and wherein determining the level of C1q protein comprises detecting and quantifying the level of a C1q fragment peptide.

3. The method of claim 1, wherein if the sample tests negative for C4 protein and tests positive for C1-INH protein and C1q protein, the subject is suffering from hereditary angioedema type II.

4. The method of claim 1, wherein if the sample tests negative for C4 protein and C1-NH protein and tests positive for C1q protein, the subject is suffering from hereditary angioedema type I.

5. The method of claim 1, wherein the method is a method for differentiating between hereditary angioedema type I and hereditary angioedema type II.

6.-8. (canceled)

9. The method of claim 2, wherein the C4 fragment peptide, the C1-INH fragment peptide and/or the C1q fragment peptide is prepared from the sample by a protease digest or a peptidase digest.

10. The method of claim 9, wherein the protease is selected from the group comprising Arg-C, Asp-N, Asp-N(N-terminal Glu), BNPS or NCS/urea, Caspase-1, Caspase-10, Caspase-2, Caspase-3, Caspase-4, Caspase-5, Caspase-6, Caspase-7, Caspase-8, Caspase-9, Chymotrypsin, Chymotrypsin (low specificity), Clostripain, CNBr, CNBr (methyl-Cys), CNBr (with acids), Enterokinase, Factor Xa, Formic acid, Glu-C (AmAc buffer, Glu-C (Phos buffer), Granzyme B, HRV3C protease, Hydroxylamine, Iodosobenzoic acid, Lys-C, Lys-N, Lys-N(Cys modified), Mild acid hydrolysis, NBS (long exposure), NBS (short exposure), NTCB, Pancreatic elastase, Pepsin A, Pepsin A (low specificity), Prolyl endopeptidase, Proteinase K, TEV protease, Thermolysin, Thrombin and trypsin.

11. (canceled)

12. The method claim 2, wherein the C4 fragment peptide is selected from the group consisting of

| Peptide | Sequence (N-terminus → C-terminus) |
|---|---|
| C4Alpha_[1006-1008] | LPR |
| C4Alpha_[1009-1026] | GCGEQTMIYLAPTLAASR |
| C4Alpha_[1009- | GCGEQTMIYLAPTLAASR |
| 1026]_Cys_CAM: 1010 | |
| C4Alpha_[1027-1030] | YLDK |
| C4Alpha_[1031-1042] | TEQWSTLPPETK |
| C4Alpha_[1043-1051] | DHAVDLIQK |
| C4Alpha_[1052-1055] | GYMR |
| C4Alpha_[1062-1072] | ADGSYAAWLSR |
| C4Alpha_[1073-1084] | GSSTWLTAFVLK |
| C4Alpha_[1085-1099] | VLSLAQEQVGGSPEK |
| C4Alpha_[1100-1126] | LQETSNWLLSQQQADGSFQDLSPVIHR |
| C4Alpha_[1168-1174] | VEASISK |
| C4Alpha_[1175-1182] | ASSFLGEK |
| C4Alpha_[1183-1204] | ASAGLLGAHAAAITAYALTLTK |
| C4Alpha_[1211-1248] | GVAHNNLMAMAQETGDNLYWGSVTGSQSNAVSP |
| | TPAPR |
| C4Alpha_[1249-1278] | NPSDPMPQAPALWIETTAYALLHLLLHEGK |
| C4Alpha_[1279-1291] | AEMADQAAAWLTR |
| C4Alpha_[1292-1300] | QGSFQGGFR |
| C4Alpha_[1301-1325] | STQDTVIALDALSAYWIASHTTEER |
| C4Alpha_[1326-1336] | GLNVTLSSTGR |
| C4Alpha_[1337-1340] | NGFK |
| C4Alpha_[1341-1349] | SHALQLNNR |
| C4Alpha_[1350-1352] | QIR |
| C4Alpha_[1353-1365] | GLEEELQFSLGSK |
| C4Alpha_[1370-1375] | VGGNSK |
| C4Alpha_[1383-1390] | TYNVLDMK |
| C4Alpha_[1391-1404] | NTTCQDLQIEVTVK |
| C4Alpha_[1391- | NTTCQDLQIEVTVK |
| 1404]_Cys_CAM: 1394 | |
| C4Alpha_[1405-1428] | GHVEYTMEANEDYEDYEYDELPAK |
| C4Alpha_[1429-1446] | DDPDAPLQPVTPLQLFEG |
| C4Alpha_[680-685] | NVNFQK |
| C4Alpha_[686-690] | AINEK |
| C4Alpha_[691-700] | LGQYASPTAK |
| C4Alpha_[702- | CCQDGVTR |
| 709]_Cys_CAM: 702, 703 | |
| C4Alpha_[710-714] | LPMMR |
| C4Alpha_[715- | SCEQR |
| 719]_Cys_CAM: 716 | |
| C4Alpha_[723-729] | VQQPDCR |
| C4Alpha_[723- | VQQPDCR |
| 729]_Cys_CAM: 728 | |
| C4Alpha_[730- | EPFLSCCQFAESLR |
| 743]_Cys_CAM: 735, 736 | |
| C4Alpha_[750-756] | GQAGLQR |
| C4Alpha_[757-775] | ALEILQEEDLIDEDDIPVR |
| C4Alpha_[776-785] | SFFPENWLWR |
| C4Alpha_[786-791] | VETVDR |
| C4Alpha_[792-815] | FQILTLWLPDSLTTWEIHGLSLSK |
| C4Alpha_[818-828] | GLCVATPVQLR |
| C4Alpha_[818- | GLCVATPVQLR |
| 828]_Cys_CAM: 820 | |
| C4Alpha_[832-838] | EFHLHLR |
| C4Alpha_[846-861] | FEQLELRPVLYNYLDK |
| C4Alpha_[862-912] | NLTVSVHVSPVEGLCLAGGGGLAQQVLVPAGSARP |
| | VAFSVVPTAATAVSLK |
| C4Alpha_[862- | NLTVSVHVSPVEGLCLAGGGGLAQQVLVPAGSARP |
| 912]_Cys_CAM: 876 | VAFSVVPTAATAVSLK |
| C4Alpha_[913-916] | VVAR |
| C4Alpha_[917-929] | GSFEFPVGDAVSK |
| C4Alpha_[936-941] | EGAIHR |
| C4Alpha_[942-954] | EELVYELNPLDHR |
| C4Alpha_[957-979] | TLEIPGNSDPNMIPDGDFNSYVR |
| C4Alpha_[980-1005] | VTASDPLDTLGSEGALSPGGVASLLR |
| C4Beta_[105-118] | GPEVQLVAHSPWLK |
| C4Beta_[119-123] | DSLSR |
| C4Beta_[124-137] | TTNIQGINLLFSSR |
| C4Beta_[139-155] | GHLFLQTDQPIYNPGQR |
| C4Beta_[158-159] | YR |
| C4Beta_[160-166] | VFALDQK |
| C4Beta_[167-185] | MRPSTDTITVMVENSHGLR |
| C4Beta_[190-214] | EVYMPSSIFQDDFVIPDISEPGTWK |
| C4Beta_[219-234] | FSDGLESNSSTQFEVK |
| C4Beta_[23-48] | LLLFSPSVVHLGVPLSVGVQLQDVPR |
| C4Beta_[236-244] | YVLPNFEVK |
| C4Beta_[245-269] | ITPGKPYILTVPGHLDEMQLDIQAR |
| C4Beta_[270-283] | YIYGKPVQGVAYVR |
| C4Beta_[284-292] | FGLLDEDGK |
| C4Beta_[294-297] | TFFR |
| C4Beta_[298-304] | GLESQTK |
| C4Beta_[305-316] | LVNGQSHISLSK |
| C4Beta_[326-337] | LNMGITDLQGLR |
| C4Beta_[338-373] | LYVAAAIIESPGGEMEEAELTSWYFVSSPFSLDLSK |
| C4Beta_[392-404] | EMSGSPASGIPVK |
| C4Beta_[405-459] | VSATVSSPGSVPEVQDIQQNTDGSGQVSIPIIIPQTISE |
| | LQLSVSAGSPHPAIAR |
| C4Beta_[460-484] | LTVAAPPSGGPGFLSIERPDSRPPR |
| C4Beta_[485-494] | VGDTLNLNLR |
| C4Beta_[49-53] | GQVVK |
| C4Beta_[495-512] | AVGSGATFSHYYYMILSR |
| C4Beta_[513-520] | GQIVFMNR |
| C4Beta_[521-523] | EPK |
| C4Beta_[525-559] | TLTSVSVFVDHHLAPSFYFVAFYYHGDHPVANSLR |
| C4Beta_[560-570] | VDVQAGACEGK |
| C4Beta_[560- | VDVQAGACEGK |
| 570]_Cys_CAM: 567 | |
| C4Beta_[571-579] | LELSVDGAK |
| C4Beta_[580-582] | QYR |
| C4Beta_[583-588] | NGESVK |
| C4Beta_[589-614] | LHLETDSLALVALGALDTALYAAGSK |
| C4Beta_[60-63] | NPSR |
| C4Beta_[615-623] | SHKPLNMGK |
| C4Beta_[624-664] | VFEAMNSYDLGCGPGGGDSALQVFQAAGLAFSDG |
| | DQWTLSR |
| C4Beta_[624- | VFEAMNSYDLGCGPGGGDSALQVFQAAGLAFSDG |
| 664]_Cys_CAM: 635 | DQWTLSR |
| C4Beta_[64-71] | NNVPCSPK |
| C4Beta_[64- | NNVPCSPK |
| 71]_Cys_CAM: 68 | |
| C4Beta_[667-671] | LSCPK |
| C4Beta_[667- | LSCPK |
| 671]_Cys_CAM: 669 | |
| C4Beta_[72-80] | VDFTLSSER |
| C4Beta_[81-92] | DFALLSLQVPLK |
| C4Beta_[93-95] | DAK |
| C4Beta_[96-104] | SCGLHQLLR |
| C4Beta_[96- | SCGLHQLLR |
| 104]_Cys_CAM: 97 | |
| C4Gamma_[1458-1465] | VVEEQESR |
| C4Gamma_[1466-1474] | VHYTVCIVVR |
| C4Gamma_[1466- | VHYTVCIVVR |
| 1474]_Cys_CAM: 1471] | |
| C4Gamma_[1475-1477] | NGK |
| C4Gamma_[1478-1498] | VGLSGMAIADVTLLSGFHALR |
| C4Gamma_[1499-1503] | ADLEK |
| C4Gamma_[1504-1510] | LTSLSDR |
| C4Gamma_[1511-1533] | YVSHFETEGPHVLLYFDSVPTSR |
| C4Gamma_[1534-1564] | ECVGFEAVQEVPVGLVQPASATLYDYYNPER |
| C4Gamma_[1534- | ECVGFEAVQEVPVGLVQPASATLYDYYNPER |
| 1564]_Cys_CAM: 1535 | |
| C4Gamma_[1566-1575] | CSVFYGAPSK |
| C4Gamma_[1566- | CSVFYGAPSK |
| 1575]_Cys_CAM: 1566 | |
| C4Gamma_[1578-1594] | LLATLCSAEVCQCAEGK |
| C4Gamma_[1578- | LLATLCSAEVCQCAEGK |
| 1594]_Cys_CAM: 1583, | |
| 1588, 1590 | |
| C4Gamma_[1595-1597] | CPR |
| C4Gamma_[1595- | CPR |
| 1597]_Cys_CAM: 1595 | |
| C4Gamma_[1601-1604] | ALER |
| C4Gamma_[1616-1622] | FACYYPR |
| C4Gamma_[1616- | FACYYPR |
| 1622]_Cys_CAM: 1618 | |
| C4Gamma_[1623-1630] | VEYGFQVK |
| C4Gamma_[1631-1633] | VLR |
| C4Gamma_[1638-1641] | AAFR |
| C4Gamma_[1642-1646] | LFETK |
| C4Gamma_[1656-1658] | DVK |
| C4Gamma_[1659-1665] | AAANQMR |
| C4Gamma_[1671-1674] | ASCR |
| C4Gamma_[1677-1681] | LEPGK |
| C4Gamma_[1682-1716] | EYLIMGLDGATYDLEGHPQYLLDSNSWIEEMPSER |
| C4Gamma_[1720-1722] | STR |
| C4Gamma_[1725-1744] | AACAQLNDFLQEYGTQGCQV |
| C4Gamma_[1725- | AACAQLNDFLQEYGTQGCQV |
| 1744]_Cys_CAM: 1727, | |
| 1742 | |

13. The method of claim 8, wherein the C4 fragment is selected from the group consisting of C4Beta[571-579], C4Alpha[680-685], C4Alpha[786-791], C4Beta[294-297] and C4Gamma[1638-1641].

14. The method of claim 2, wherein the C1-INH fragment peptide is selected from the group consisting of

| Peptide | Sequence (N-terminus → C-terminus) |
|---|---|
| SerpinG1_[202-211] | DFTCVHQALK |
| SerpinG1_[202- | DFTCVHQALK |
| 211]_Cys_CAM: 205 | |
| SerpinG1_[212-216] | GFTTK |
| SerpinG1_[217-233] | GVTSVSQIFHSPDLAIR |
| SerpinG1_[23-40] | NPNATSSSSQDPESLQDR |
| SerpinG1_[234-241] | DTFVNASR |
| SerpinG1_[242-249] | TLYSSSPR |
| SerpinG1_[250-268] | VLSNNSDANLELINTWVAK |
| SerpinG1_[269-273] | NTNNK |
| SerpinG1_[274-276] | ISR |
| SerpinG1_[277-286] | LLDSLPSDTR |
| SerpinG1_[301-306] | TTFDPK |
| SerpinG1_[310-316] | MEPFHFK |
| SerpinG1_[322-328] | VPMMNSK |
| SerpinG1_[330-341] | YPVAHFIDQTLK |
| SerpinG1_[344-364] | VGQLQLSHNLSLVILVPQNLK |
| SerpinG1_[367-380] | LEDMEQALSPSVFK |
| SerpinG1_[381-385] | AIMEK |
| SerpinG1_[386-390] | LEMSK |
| SerpinG1_[391-400] | FQPTLLTLPR |
| SerpinG1_[403-415] | VTTSQDMLSIMEK |
| SerpinG1_[41-44] | GEGK |
| SerpinG1_[416-466] | LEFFDFSYDLNLCGLTEDPDLQVSAMQHQTVLELTETGV |
| | EAAAASAISVAR |
| SerpinG1_[467-487] | TLLVFEVQQPFLFVLWDQQHK |
| SerpinG1_[488-494] | FPVFMGR |
| SerpinG1_[495-499] | VYDPR |
| SerpinG1_[53-77] | MLFVEPILEVSSLPTTNSTTNSATK |

15. The method of claim 10, wherein the C1-INH fragment peptide is selected from the group consisting of SerpinG1 [242-249] and SerpinG1 [391-400].

16. The method of claim 2, wherein the C1q fragment peptide is selected from the group consisting of

| Peptide | Sequence (N-terminus → C-terminus) |
|---|---|
| C1q-A_[104-110] | GSPGNIK |
| C1q-A_[111-121] | DQPRPAFSAIR |
| C1q-A_[123-150] | NPPMGGNVVIFDTVITNQEEPYQNHSGR |
| C1q-A_[151-180] | FVCTVPGYYYFTFQVLSQWEICLSIVSSSR |
| C1q-A_[151- | FVCTVPGYYYFTFQVLSQWEICLSIVSSSR |
| 180]_Cys_CAM: 153, 172 | |
| C1q-A_[186-195] | SLGFCDTTNK |
| C1q-A_[186- | SLGFCDTTNK |
| 195]_Cys_CAM: 190 | |
| C1q-A_[196-219] | GLFQVVSGGMVLQLQQGDQVWVEK |
| C1q-A_[224-245] | GHIYQGSEADSVFSGFLIFPSA |
| C1q-A_[23-27] | EDLCR |
| C1q-A_[28-32] | APDGK |
| C1q-A_[34-41] | GEAGRPGR |
| C1q-A_[49-60] | GEQGEPGAPGIR |
| C1q-A_[82-94] | VGYPGPSGPLGAR |
| C1q-B_[118-121] | ATQK |
| C1q-B_[137-141] | DQTIR |
| C1q-B_[160-163] | FTCK |
| C1q-B_[164-177] | VPGLYYFTYHASSR |
| C1q-B_[178-186] | GNLCVNLMR |
| C1q-B_[178- | GNLCVNLMR |
| 186]_Cys_CAM: 181 | |
| C1q-B_[194-215] | VVTFCDYAYNTFQVTTGGMVLK |
| C1q-B_[194- | VVTFCDYAYNTFQVTTGGMVLK |
| 215]_Cys_CAM: 198 | |
| C1q-B_[216-229] | LEQGENVFLQATDK |
| C1q-B_[230-253] | NSLLGMEGANSIFSGFLLFPDMEA |
| Clq-B_[28-59] | QLSCTGPPAIPGIPGIPGTPGPDGQPGTPGIK |
| C1q-B_[28- | QLSCTGPPAIPGIPGIPGTPGPDGQPGTPGIK |
| 59]_Cys_CAM: 31 | |
| C1q-B_[63-77] | GLPGLAGDHGEFGEK |
| C1q-B_[78-88] | GDPGIPGNPGK |
| C1q-B_[93-98] | GPMGPK |
| C1q-B_[99-110] | GGPGAPGAPGPK |
| C1q-C_[118-126] | FQSVFTVTR |
| C1q-C_[127-139] | QTHQPPAPNSLIR |
| C1q-C_[140-157] | FNAVLTNPQGDYDTSTGK |
| C1q-C_[162-184] | VPGLYYFVYHASHTANLCVLLYR |
| C1q-C_[162- | VPGLYYFVYHASHTANLCVLLYR |
| 184]_Cys_CAM: 179 | |
| C1q-C_[189-198] | VVTFCGHTSK |
| C1q-C_[189- | VVTFCGHTSK |
| 198]_Cys_CAM: 193 | |
| C1q-C_[199-210] | TNQVNSGGVLLR |
| C1q-C_[211-245] | LQVGEEVWLAVNDYYDMVGIQGSDSVFSGFLLFPD |
| C1q-C_[29-47] | NTGCYGIPGMPGLPGAPGK |
| C1q-C_[29- | NTGCYGIPGMPGLPGAPGK |
| 47]_Cys_CAM: 32 | |
| C1q-C_[48-57] | DGYDGLPGPK |
| C1q-C_[58-69] | GEPGIPAIPGIR |
| C1q-C_[76-86] | GEPGLPGHPGK |
| C1q-C_[87-113] | NGPMGPPGMPGVPGPMGIPGEPGEEGR |

17. The method of claim 12, wherein the C1q fragment peptide is selected from the group consisting of C1qBeta[178-186] and C1qBeta[63-77].

18. The method of claim 9, wherein
the cut-off value for C4 fragment peptide C4Beta[571-579] is 500 ng/ml;
the cut-off value for C4 fragment peptide C4Alpha[680-685] is 260 ng/ml;
the cut-off value for C4 fragment peptide C4Alpha[786-791] is 100 ng/ml;
the cut-off value for C4 fragment peptide C4Beta[294-297] is 201 ng/ml; and
the cut-off value for C4 fragment peptide C4Gamma[1638-1641] is 920 ng/ml.

19. The method of claim 11, wherein
the cut-off value for C1-INH fragment peptide SerpinG1[242-249] is 835 ng/ml, and
the cut-off value for C1-INH fragment peptide SerpinG1[391-400] is 392 ng/ml.

20. The method of claim 13, wherein
the cut-off value for C1q fragment peptide C1qBeta[178-186] is 800 ng/ml, and
the cut-off value for C1q fragment peptide and C1qBeta[63-77] is 1690 ng/ml.

21. (canceled)

22. The method of claim 1, wherein mass spectrometry is selected from the group comprising SELDI MS, MALDI MS, ESI MS, DESI MS and ion mobility MS.

23. The method of claim 1, wherein mass spectrometry uses an analyzer selected from the group comprising Triple Quad, ToF, QToF, ion trap, orbitrap, ion mobility and any combination thereof.

24. The method of claim 1, wherein spectrometric analysis comprises or uses MS/MS, MRM, SRM or any combination thereof.

25. A kit suitable for use in a method for differential diagnosis of hereditary angioedema, wherein the kit comprises at least one element selected from the group comprising an interaction partner of one biomarker, one biomarker, instructions of use for the kit, and one or more container, wherein the biomarker is selected from the group comprising C4 protein, a fragment peptide of C4 protein, C1-INH protein, a fragment peptide of C1-INH protein, C1q protein and a fragment peptide of C1q.
